(19) United States
(12) Patent Application Publication
(10) Pub. No.: US 2023/0332255 A1
(43) Pub. Date: Oct. 19, 2023

(54) CRISPR-CAS-BASED DETECTION OF SARS-COV-2 USING RECOMBINASE POLYMERASE AMPLIFICATION

(71) Applicants: The Regents of the University of California, Oakland, CA (US); Mammoth Biosciences, Inc., Brisbane, CA (US)

(72) Inventors: Charles Chiu, Oakland, CA (US); Xian Ding Deng, Oakland, CA (US); Janice Sha Chen, Brisbane, CA (US); James Broughton, Brisbane, CA (US); Matthew Verosloff, Brisbane, CA (US)

(21) Appl. No.: 18/014,167

(22) PCT Filed: Jul. 2, 2021
(86) PCT No.: PCT/US2021/040338

§ 371 Date: Jan. 2, 2023

Related U.S. Application Data
(60) Provisional application No. 63/048,060, filed on Jul. 3, 2020.

(51) Int. Cl. C12Q 1/70; C12Q 1/6844

(52) U.S. Cl. CPC C12Q 1/701; C12Q 1/6844

(57) ABSTRACT

The disclosure provides for CRISPR-Cas-based detection of SARS-CoV-2 using recombinase polymerase amplification, and uses thereof.

### CROSS REFERENCE TO RELATED APPLICATIONS

This application claims priority to U.S. Provisional Application Ser. No. 63/048,060, filed on Jul. 3, 2020, the disclosures of which are incorporated herein by reference.

### STATEMENT OF GOVERNMENT SUPPORT

This invention was made with government support under Grant Number R33-AI120977 awarded by the National Institutes of Health. The government has certain rights in the invention.

### TECHNICAL FIELD

The disclosure provides for CRISPR-Cas-based detection of SARS-CoV-2 using recombinase polymerase amplification.

### INCORPORATION BY REFERENCE OF SEQUENCE LISTING

Accompanying this filing is a Sequence Listing entitled “Sequence-Listing_ST25.txt”, created on Jul. 2, 2021 and having 48,739 bytes of data, machine formatted on IBM-PC, MS-Windows operating system. The sequence listing is hereby incorporated herein by reference in its entirety for all purposes.

### BACKGROUND

Over the past 40 years, there have been recurrent large-scale epidemics from novel emerging viruses, including human immunodeficiency virus (HIV), SARS and MERS coronaviruses, 2009 pandemic influenza H1N1 virus, Ebola virus (EBOV), Zika virus (ZIKV), and most recently SARS-CoV-2. All of these epidemics presumably resulted from an initial zoonotic animal-to-human transmission event, with either clinically apparent or occult spread into vulnerable human populations. Each time, a lack of rapid, accessible, and accurate molecular diagnostic testing has hindered the public health response to the emerging viral threat.

### SUMMARY

An outbreak of SARS-CoV-2 began in Wuhan, China in December 2019. COVID-19, the disease associated with SARS-CoV-2 infection, has rapidly spread to produce a global pandemic. Provided herein is the development of a rapid (<40 min), easy-to-implement and accurate CRISPR-Cas12-based assay for detection of SARS-CoV-2 from clinical samples using recombinase polymerase amplification. The CRISPR-Cas12-based detection assay disclosed herein was validated against contrived reference and clinical respiratory samples from patients in the United States. As highlighted in the Figures, the CRISPR-based detection assay described herein provides a visual and faster alternative to the US Centers for Disease Control and Prevention SARS-CoV-2 real-time RT-PCR assay.

In a particular embodiment, the disclosure provides for a recombinase polymerase amplification (RPA) method to detect SARS-CoV-2 in a sample, comprising: performing a RT-RPA reaction to generate isothermal amplification products by incubating at a temperature of about 40-42° C. a first reaction mixture comprising reverse transcriptase, extracted RNA from a sample, primers that are specific to a gene of SARS-CoV-2, and recombinase and polymerase enzymes, wherein the gene is the N-gene, or E-gene of SARS-CoV-2; performing a trans-cleavage assay by incubating at a temperature of about 37° C. a second reaction mixture comprising the amplification products from the previous step, a programmable nuclease that has been complexed with a gRNA specific to corresponding gene sequences of SARS-CoV-2, and a single-stranded detector nucleic acid comprising a detection moiety, wherein the detector nucleic acid is trans-cleaved by the programmable nuclease if SARS-CoV-2 gene amplification products are present in the second reaction mixture; and detecting whether SARS-CoV-2 is the sample based upon detecting trans-cleaved detector nucleic acid fragments. In another embodiment, the recombinase and polymerase enzymes include one or more of the following T4 UvsX protein, T4 UvsY protein, T4 gp32, Bacillus subtilis DNA polymerase I and Staphylococcus aureus polymerase. In yet another embodiment, the first reaction mixture is incubated at 40-42° C. for 20-30 minutes. In yet a further embodiment, the primers target the N2 region in the N gene of SARS-CoV-2. In a certain embodiment, the primers have the sequences of (1) or (2): (1) GAAGAGACAGGTACGTTAATAGTTAATAGCGT (SEQ ID NO:5) and/or CGTTAACAATATTGCAGCAGTACGCACACA (SEQ ID NO:6); or (2) ACAAGGAACTGATTACAAACATTGGCCGCAAA (SEQ ID NO:7) and/or TTCCATG CCAATGCGCGACATTCCGAAGAA (SEQ ID NO:8). In another embodiment, the RNA is extracted from an environmental sample or a sample from a subject. In a certain embodiment, the sample is a nasopharyngeal and/or oropharyngeal swab sample obtained from a human patient. In yet another embodiment, the RNA is extracted from the sample using a commercially available RNA extraction kit. In another embodiment, RT-RPA reaction and the trans-cleavage assay are performed as a single-tube reaction. In yet a further embodiment, the programmable nuclease is a Cas12 nuclease, a Cas13 nuclease, or a Cas14 nuclease. In another embodiment, the programmable nuclease is a Cas12a nuclease. In yet another embodiment, the polypeptide sequence of the programmable nuclease has at least 85% sequence identity to SEQ ID NO:1, 2, 3, or 4. In a certain embodiment, the polypeptide sequence of the programmable nuclease has at least 95% sequence identity to SEQ ID NO:4. In another embodiment, the gRNA is specific to the N-gene, E-gene of SARS-CoV-2, or Rnase P gene of human matrix as internal control. In yet another embodiment, the gRNA is specific to the N-gene of SARS-CoV-2 and comprises the sequence UAAUUUCUACUAAGUGUAGAUCCCCCAGCGCUUCAGCGUUC (SEQ ID NO:9). In a further embodiment, the gRNA is specific to the E-gene of SARS-CoV-2 and comprises the sequence of (c) or (d): (c) UAAUUUCUACUAAGUGUAGAUUUGCUUUCGUGGUAUUCUUG (SEQ ID NO:10); or (d) UAAUUUCUACUAAGUGUAGAUGUGGUAUUCUUGCUAGUUAC (SEQ ID NO:11). In yet a further embodiment, the detector nucleic acid comprises a fluorescence detection moiety. In another embodiment, the fluorescence detection molecule is fluorescein or a chemical derivative thereof. In yet another embodiment, the detector nucleic acid comprises a biotin moiety. In a further embodiment, the detector nucleic acid comprises the sequence of (e) or (f): (e) 5′-(FAM)-TTATTATT-(BHQ-1)-3′, wherein FAM is fluorescein and wherein BHQ-1 is a black hole quencher dye; or (f) 5′-(FAM)-TTATTATT-(Bio)-3′, wherein FAM is fluorescein and wherein Bio is biotin. In yet a further embodiment, the trans-cleaved detector nucleic acid fragments are detected using a lateral flow assay. In another embodiment, the trans-cleaved detector nucleic acid fragments are detected using a microplate reader.

The details of one or more embodiments of the disclosure are set forth in the accompanying drawings and the description below. Other features, objects, and advantages will be apparent from the description and drawings, and from the claims.

### BRIEF DESCRIPTION OF DRAWINGS

FIG. 1A-E presents a genome map showing primers, probes and gRNAs and SARS-CoV-2 detection work-up. (A) Visualization of primers and probes on the SARS-CoV-2 genome. RPA primers are indicated by black rectangles. (B) gRNA specificity. Cas12 gRNAs are programmed to specifically target SARS-CoV-2 or broadly detect related coronavirus strains. The N gene gRNA used in the assay (left) was specific for SARS-CoV-2, whereas the E gene gRNA was able to detect three SARS-like coronavirus strains (right). A separate N gene gRNA designed to target SARS-CoV and a bat coronavirus failed to detect SARS-CoV-2 (middle). (C) The minimum equipment needed to run the protocol. With appropriate biosafety level 2 requirements, the minimum equipment required to run the protocol following RNA extraction includes Eppendorf tubes with reagents, heat blocks or water bath (37° C. and 42° C.), nuclease-free water, pipettes and tips and lateral flow strips. (D) Schematic of SARS-CoV-2 DETECTR workflow. Conventional RNA extraction can be used as an input to DETECTR (RPA preamplification and Cas12-based detection for E gene, N gene and RNase P), which is visualized by a fluorescent reader or lateral flow strip. (E) Lateral flow strip assay readout. A positive result requires detection of at least one of the two SARS-CoV-2 viral gene targets (N gene or E gene, as indicated in the interpretation matrix). QC, quality control.

FIG. 2 provides for detection of SARS-CoV-2 in contrived nasal swab samples (in vitro SARS-CoV-2 RNA spiked into negative nasal swab matrix) using lateral flow strip (E-gene: 100 cp/μl; N-gene: 50 cp/μl).

FIG. 3 presents the results of in-vitro transcribed (IVT) RNA of N2 gene of SARS-CoV-2: RT-RPA followed by fluorescence reading using Synergy H4 machine D1, 1000 cp/μl; D2, 500 cp/μl; D3, 250 cp/μl; D4, 100 cp/μl; D5, 50 cp/μl; D6, 25 cp/μl; D7, 10 cp/μl, all above with strong signal. D8, 1 cp/μl, no signal; D9, positive control, strong signal; D10, negative control, no signal. E1-10 are empty wells for purposes of comparison. RT-RPA can detect SARS-CoV-2 at 10 copies per μL.

FIG. 4 provides RT-RPA Triplicate runs of E gene of SARS-CoV-2: A1-3: 10≡copies/μl, triplicate, strong signal; B1-3: 10∝cp/μl, triplicate, strong signal; C1-3: 10\hat{ }cp/μl, triplicate, strong signal; D1-3: 100 cp/μl, triplicate, strong signal; E1-3: 10 cp/μl, triplicate, strong signal; F1-3: 5 cp/μl; 2 out of 3 no signal; G1-3: 1 cp/μl; 1 out of 3 no signal. Approximate limit of detection is 10 cp/μl (3 of 3, 100% positive).

FIG. 5 presents the results of in-vitro transcribed (IVT) RNA of N2 gene of SARS-CoV-2: RT-RPA with three different reverse transcriptases (SSII, M71 MuLV, RevertAid) using two different primers (N-gene, RNAse P) followed by fluorescence reading of a Cas12-based DETECTR assay (N-gene: 5000 cp, 500 cp, 50 cp, 5 cp, 0 cp).

FIG. 6 presents the results of in-vitro transcribed (IVT) RNA of N2 gene of SARS-CoV-2: RT-RPA with two different primers (N-gene, RNAse P) followed by fluorescence reading of a Cas12-based DETECTR assay using different guide nucleic acids (1763: N-gene, 1764: E-gene).

FIG. 7 presents the results of in-vitro transcribed (IVT) RNA of N2 gene of SARS-COV2: RT-RPA with different dNTP concentrations (1.6 mM, 1.4 mM, 1.2 mM, 1 mM, 0.8 mM, 0.6 mM, 0.4 mM, 0 mM) followed by fluorescence reading of a Cas12-based DETECTR assay using different volumes of RT-RPA reaction (0.5 μl, 1 μl, 5 μl, 10 μl, 15 μl).

The accompanying drawings, which are incorporated into and constitute a part of this specification, illustrate one or more embodiments of the disclosure and, together with the detailed description, serve to explain the principles and implementations of the disclosure.

### DETAILED DESCRIPTION

As used herein and in the appended claims, the singular forms “a,” “an,” and “the” include plural referents unless the context clearly dictates otherwise. Thus, for example, reference to “a Cas-12 based assay” includes a plurality of such Cas-12 based assays and reference to “the RPA primer” includes reference to one or more RPA primers, and so forth.

Also, the use of “or” means “and/or” unless stated otherwise. Similarly, “comprise,” “comprises,” “comprising,” “include,” “includes,” “including,” “have,” “haves,” and “having” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

The disclosure provides various compositions and method of use thereof for assaying for and detecting SARS-CoV-2 in a sample. In particular, the various methods, reagents, and devices disclosed herein use a programmable nuclease complexed with guide nucleic acid sequences to detect the presence or absence of, and/or quantify the amount of, nucleic acids from SARS-CoV-2 in a sample. Disclosed herein is a CRISPR (clustered regularly interspaced short palindromic repeats)-Cas12 based assay for detection of SARS-CoV-2, in patient samples in approximately 30 minutes. The detection assays disclose herein feature low coast, portability, and accurate detection of SARS-CoV-2 and may be performed using commercially available reagents and devices. The compositions and method disclosed herein may be used to detect the presence or absence of SARS-CoV-2 in a sample, such as a sample obtained from a subject or an environmental sample. In a further embodiment, a subject may be diagnosed with COVID-19 by detecting the presence of SARS-CoV-2 in the subject's sample using the methods of the disclosure. SARS-CoV-2-Dectection assays disclosed herein use recombinase polymerase amplification (isothermal amplification) of RNA, followed by Cas12 detection of predefined coronavirus sequences, followed by cleavage of a reporter molecule to detect the presence of SARS-CoV-2. In a particular embodiment, the SARS-CoV-2-Dectection assays may target the E (envelope) genes or N (nucleoprotein) genes of SARS-CoV-2. RPA may be used to amplify one or more regions of an N-gene and/or E-gene of SARS-CoV-2. Also disclosed herein are primer sets specifically designed for RPA amplification of one or more regions of an N-gene and/or E-gene of SARS-CoV-2. Any nucleic acid of SARS-CoV-2 can be assayed using the compositions and methods of the disclosure. In a certain embodiment, the target nucleic acid comprises the N-gene and/or E-gene of SARS-CoV-2 that is assayed by using the compositions and methods of the disclosure. Further disclosed herein are guide nucleic acids (gRNAs) for the specific detection of the N-gene of SARS-CoV-2. Also disclosed herein are guide nucleic acids (gRNAs) for the specific detection of the E-gene of SARS-CoV-2.

As used herein, the terms “guide polynucleotide,” “guide sequence,” or “guide RNA” as can refer to any polynucleotide sequence having sufficient complementarity with a target polynucleotide sequence to hybridize with the target sequence and direct sequence-specific binding of a CRISPR complex to the target sequence. The degree of complementarity between a guide polynucleotide and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences known in the art. A guide CRIPSR RNA (crRNA) can be a guide polynucleotide or guide RNA and is used interchangeably herein.

The term “pharmaceutically acceptable” as in pharmaceutically acceptable salt or pharmaceutically acceptable counter ion, refers to compounds, salts, or ions that are tolerated by a subject for topical, or internal use.

A “subject” generally refers to mammals such as human patients and non-human primates, as well as experimental animals such as rabbits, rats, and mice, and other animals. Animals include all vertebrates, e.g., mammals and non-mammals, such as sheep, dogs, cows, chickens, amphibians, and reptiles.

All publications mentioned herein are incorporated herein by reference in full for the purpose of describing and disclosing the methodologies, which are described in the publications, which might be used in connection with the description herein. The publications discussed above and throughout the text are provided solely for their disclosure prior to the filing date of the present application. Nothing herein is to be construed as an admission that the inventors are not entitled to antedate such disclosure by virtue of prior disclosure. Moreover, with respect to similar or identical terms found in the incorporated references and terms expressly defined in this disclosure, the term definitions provided in this disclosure will control in all respects.

In early January 2020, a cluster of cases of pneumonia from a new coronavirus, SARS-CoV-2 (with the disease referred to as COVID-19), was reported in Wuhan, China. This outbreak has spread rapidly, with over 182 million reported cases and more than 3 million deaths worldwide as of June 2021. Person-to-person transmission from infected individuals with no or mild symptoms has been reported. Assays using quantitative RT-PCR (qRT-PCR) approaches for detection of the virus in 4-6 h have been developed by several laboratories, including an emergency use authorization (EUA)-approved assay developed by the US Centers for Disease Control and Prevention (CDC). However, the typical turnaround time for screening and diagnosing patients with suspected SARS-CoV-2 has been >24 h, given the need to ship samples overnight to reference laboratories. Although serology tests are rapid and require minimal equipment, their utility may be limited for diagnosis of acute SARS-CoV-2 infection, because it can take several days to weeks following symptom onset for a patient to mount a detectable antibody response. To accelerate clinical diagnostic testing for COVID-19 in the United States, on 28 Feb. 2020, the US Food and Drug Administration (FDA) permitted individual clinically licensed laboratories to report the results of in-house-developed SARS-CoV-2 diagnostic assays while awaiting results of an EUA submission for approval.

CRISPR is a biotechnological technique well-known for its use in gene editing. Notably, CRISPR has been recently used for the in vitro detection of nucleic acids, thereby emerging as a powerful and precise tool for molecular diagnosis. Within the CRISPR-Cas effector family, Cas12 is an RNA-guided DNase belonging to the class II type V-A system that induces indiscriminate single-stranded DNA (ssDNA) collateral cleavage after target recognition. This leads to the degradation of ssDNA reporters that, emit a fluorescence signal on cleavage or alternatively, could be detected on a paper strip (by lateral flow) in a portable manner. Therefore, CRISPR-Cas12 based tools possess the potential to emerge as an in situ diagnostic tool for rapid detection of the SARS-CoV-2 virus.

The disclosure provides a CRISPR-Cas12-based assay for the detection of SARS-CoV-2 from extracted patient sample RNA. This assay performs simultaneous reverse transcription and isothermal amplification using recombinase polymerase amplification (RPA) for RNA extracted from nasopharyngeal or oropharyngeal swabs in universal transport medium (UTM), followed by Cas12 detection of predefined coronavirus sequences, after which cleavage of a reporter molecule confirms detection of the virus.

The fundamental reaction mechanism of RPA relies on a synthetically engineered adaptation of a natural cellular process called homologous recombination, a key process in DNA metabolism. The standard RPA reaction reagents comprise three proteins (recombinase, recombinase loading factor and single-stranded binding protein), which subsequently coordinate with ancillary components such as deoxyribonucleic acid (DNA) polymerase, crowding agent, energy/fuel components (e.g., adenosine triphosphate, ATP) and salt molecules to perform the RPA reaction mechanism. RPA starts with the binding of the T4 UvsX protein (recombinase), assisted by the T4 UvsY (loading factor), to the primers to form a nucleoprotein filament. The resulting complex searches for homologous sequences in duplex DNA. Once the homology is located, the complex invades the double-stranded DNA, forming a D-loop structure. One side of the D-loop is double-stranded where the primer hybridizes with the template strand, initiating a strand exchange reaction, whereas the other side of the D-loop remains single-stranded—the unwound complementary strand that is stabilized by the SSB proteins (T4 gp32). Subsequently, the recombinase disassembles from the nucleoprotein filament and becomes immediately available to initiate another strand displacement reaction with a new primer. Primer incorporation allows the DNA polymerase (Bsu or Sau) to initiate the synthesis from the free 3′-OH at the end of the primer. As the polymerization continues, the two parental strands continue to separate. Incorporation of both forward and reverse RPA primers enables strand synthesis to occur in both directions simultaneously, and ultimately results in the exponential accumulation of amplified duplex DNA, consisting of the sequence between the forward and reverse RPA primers. Unlike PCR, the length of RPA primers is relatively long (a recommended minimum of 30 nucleotides, but typically between 32 and 35 nucleotides). Shorter PCR primers (typically between 18 and 25 nucleotides) can also be used in the RPA reaction but may decrease the reaction speed and sensitivity. All the RPA reagents are available for commercial purchase through TwistDx™, a subsidiary of Abbott.

In a particular embodiment, a programmable nuclease can be used for detection of a target nucleic acid from SARS-CoV-2 in a sample (e.g., a subject's sample or an environmental sample). For example, a programmable nuclease can be complexed with a guide nucleic acid that hybridizes to a target sequence of a target nucleic acid from SARS-CoV-2. The complex can be contacted to a sample from a subject. The subject may or may not be infected with SARS-CoV-2. The target nucleic acid in the sample can be reversed transcribed and isothermally amplified using RPA. In same embodiment, the reverse transcription and isothermal amplification may be performed simultaneously. If the subject is infected with SARS-CoV-2, the guide nucleic acid hybridizes to the target nucleic acid leading to activation of programmable nuclease. Upon activation, the programmable nuclease can cleave a detector nucleic acid, wherein the detector nucleic acid comprises a detectable label attached to a polynucleotide (e.g., polydeoxyribonucleotide or polyribonucleotide). In some embodiments of the assay, upon cleavage of the polynucleotide, the detectable label emits a detectable signal, which is then captured and quantified (e.g., the detectable label is a fluorophore and the detectable signal is fluorescence). Upon detection of the detectable label, it can be determined that the sample from the subject contained target nucleic acids from SARS-Cov-2. In further embodiments, the target nucleic acid comprises the N gene or the E gene of SARS-CoV-2 and can be assayed by using the compositions and methods of the disclosure.

The compositions and method of use disclosed herein include using a programmable nuclease such as a Cas12 protein, a Cas13 protein, or a Cas14 protein to assay for, detect and/or quantify a nucleic acid from SARS-CoV-2. In a particular embodiment, a Cas12 protein, a Cas13 protein, or a Cas14 protein is used for detection of a target nucleic acid from SARS-CoV-2 in a sample from a subject. For example, a Cas12 protein, a Cas13 protein, or a Cas14 protein is complexed with a guide nucleic acid that hybridizes to a target sequence of a target nucleic acid from SARS-CoV-2. The complex can be contacted to a sample from a subject or an environmental sample. The sample may or may not comprise SARS-CoV-2. A target nucleic acid in the sample can be reverse transcribed back into RNA in any assay using a Cas12 protein, a Cas13 protein or a Cas14 protein. If the subject is infected with SARS-CoV-2, the guide nucleic acid hybridizes to the target nucleic acid or amplicon thereof leading to activation of the Cas12 protein, the Cas13 protein, or the Cas14 protein. Upon activation, the Cas12 protein, the Cas13 protein, or the Cas14 protein can cleave a detector nucleic acid, wherein the detector nucleic acid comprises a detectable label attached to the nucleic acid for cleavage by the Cas12 protein, the Cas13 protein, or the Cas14 protein. In another embodiment, upon cleavage of the detector nucleic acid, the detectable label emits a detectable signal, which can then be captured and quantified (e.g., the detectable label is a fluorophore and the detectable signal is fluorescence). Upon detection of a detectable label, it can be determined that the sample from the subject comprise the target nucleic acids from SARS-CoV-2. In yet another embodiment, the target nucleic acid comprises the N gene or the E gene of SARS-CoV-2 and can be assayed by using the compositions and methods disclosed herein.

In some embodiments, a programmable nuclease having at least 60%, at least 65%, at least 70%, at least 75%, at least 80%, at least 85%, at least 90%, at least 95%, at least 97%, at least 98%, or at least 99% sequence identity to any of SEQ ID NOs:1-3 or 4 can be used for detection of a target nucleic acid from SARS-CoV-2. For example, a programmable nuclease having at least 60%, at least 65%, at least 70%, at least 75%, at least 80%, at least 85%, at least 90%, at least 95%, at least 97%, at least 98%, or at least 99% identity sequence identity to any of SEQ ID NOs:1-3 or 4 can be complexed with a guide nucleic acid that hybridizes to a target sequence of a target nucleic acid from SARS-CoV-2. The complex can be contacted to a subject's sample or an environmental sample. The target nucleic acid of the sample can be reverse transcribed and amplified by RPA. If the subject is infected with SARS-CoV-2, the guide nucleic acid hybridizes to the target nucleic acid leading to activation of programmable nuclease having at least 60%, at least 65%, at least 70%, at least 75%, at least 80%, at least 85%, at least 90%, at least 95%, at least 97%, at least 98%, or at least 99% identity sequence identity to any of SEQ ID NOs:1-3 or 4 can cleave a detector nucleic acid, wherein the detector nucleic acid comprises a detectable label attached to a nucleic acid. In some embodiments of the assay, upon cleavage the detector nucleic acid, the detectable label emits a detectable signal, which can then be captured and/or quantified (e.g., the detectable label is a fluorophore and the detectable signal is fluorescence). Upon detection of a detectable label, it can be determined that the sample from the subject contained target nucleic acids from SARS-CoV-2. In some embodiment, the target nucleic acid comprises the N gene or the E gene of SARS-CoV-2 and can be assayed for using the compositions and methods disclosed herein.

The compositions and methods disclosed herein can be used as a companion diagnostic with medicaments used to treat SARS-CoV-2, or can be used in reagent kits, point-of-care diagnostics, or over-the-counter diagnostics. The methods of the disclosure may be used as a point-of-care diagnostic or as a lab test for detection of a target nucleic acid from SARS-CoV-2, and thereby, detection of a COVID-19 in a subject from which the sample was taken. The compositions and methods of the disclosure may be used in various sites or locations, such as in laboratories, in hospitals, in physician offices/laboratories (POLs), in clinics, at remote sites, or at home.

Also described herein are methods, reagents, and devices for detecting the presence of a target nucleic acid from SARS-CoV-2 in a sample. The methods, reagents, and devices for detecting the presence of a target nucleic acid from SARS-CoV-2 in a sample can be used as a rapid test for detecting a SARS-CoV-2 target nucleic acid of interest. In particular, provided herein are methods, reagents, and devices, wherein the test can be performed in a single system. The target nucleic acid may be a portion of a nucleic acid from SARS-CoV-2. The target nucleic acid may be a portion of an RNA or DNA or an amplicon thereof from SARS-CoV-2.

In some embodiments, programmable nucleases disclosed herein are activated by RNA or DNA to initiate trans cleavage activity of a detector nucleic acid. A programmable nuclease as disclosed herein, in some cases, binds to a target RNA to initiate trans cleavage of a detector nucleic acid, and this programmable nuclease can be referred to as an RNA-activated programmable RNA nuclease. In some instances, a programmable nuclease as disclose herein binds to a target DNA to initiate trans cleavage of a detector nucleic acid, and this programmable nuclease can be referred to as a DNA-activated programmable RNA nuclease. In some cases, a programmable nuclease as described herein is capable of being activated by a target RNA or a target DNA. For example, a Cas13 protein, such as Cas13a, disclosed herein is activated by a target RNA nucleic acid or a target DNA nucleic acid to trans-collaterally cleave RNA detector nucleic acids. In another embodiment, the Cas13 binds to a target ssDNA which initiates trans cleavage of RNA detector nucleic acid.

The detection of the target nucleic acid in the sample may indicate the presence of SARS-CoV-2 in the sample and may provide information for taking action to reduce the transmission of the disease to individuals in the disease-affected environment or near the disease-carrying individual. The detection of the target nucleic acid from SARS-CoV-2 is facilitated by a programmable nuclease. The programmable nuclease can become activated after binding of a guide nucleic acid with a target nucleic acid from SARS-CoV-2, in which the activated programmable nuclease can cleave the target nucleic acid and can have trans cleavage activity, which can also be referred to as “collateral” or “trans-collateral” cleavage.

Trans cleavage activity can be non-specific cleavage of nearby single-stranded nucleic acids by the activated programmable nuclease, such as trans cleavage of detector nucleic acids with a detection moiety. Once the detector nucleic acid is cleaved by the activated programmable nuclease, the detection moiety is released from the detector nucleic acid and generates a detectable signal that is immobilized on a support medium. Often the detection moiety is at least one of a fluorophore, a dye, a polypeptide, or a nucleic acid. Sometimes the detection moiety binds to a capture molecule on the support medium to assess the presence or level of the target nucleic acid associated with an ailment, such as a disease. The programmable nuclease can be a CRISPR-Cas (clustered regularly interspaced short palindromic repeats—CRISPR associated) nucleoprotein complex with trans cleavage activity, which can be activated by binding of a guide nucleic acid with a target nucleic acid. These assays, which leverage the trans-collateral cleavage properties of CRISPR-Cas enzymes. The assays of the disclosure can be performed in a microfluidic device or genechip.

In a particular embodiment, the disclosure further provides for Cas12 detection of a target nucleic acid from SARS-CoV-2. In this case, nucleic acids (RNA) from a sample are reverse transcribed and amplified into cDNA. Any Cas12 protein disclosed herein is complexed with a guide nucleic acid designed to hybridize to a nucleic acid sequence of the reverse transcribed and amplified DNA. In the presence of reverse transcribed and amplified DNA, Cas12 is activated to trans-collaterally cleave a detector nucleic acid, emitting a detectable signal (e.g., fluorescence).

Also described herein is a kit for detecting a target nucleic acid from SARS-CoV-2. The kit may comprise a support medium; guide nucleic acid sequences targeted to a target nucleic acid sequence from SARS-CoV-2; a programmable nuclease capable of being activated when complexed with a guide nucleic acid and a target nucleic acid from SARS-CoV-2; and a single-stranded detector nucleic acid comprising a detection moiety, wherein the detector nucleic acid is capable of being cleaved by the activated nuclease, thereby generating a first detectable signal.

A biological sample from an individual or an environmental sample can be tested to determine whether the individual has SARS-CoV-2. A sample from an individual or from an environment is applied to the reagents described herein. If the target nucleic acid from SARS-CoV-2 is present in the sample, the target nucleic acid binds to the guide nucleic acid to activate the programmable nuclease. The activated programmable nuclease cleaves the detector nucleic acid and generates a detectable signal that can be visualized, for example on a support medium. If the target nucleic acid from SARS-CoV-2 is absent in the sample or below the threshold of detection, the guide nucleic acid remains unbound, the programmable nuclease remains inactivated, and the detector nucleic acid remains uncleaved.

The method, reagents, and devices described herein may allow for detection of target nucleic acid, and in turn the disease associated with the target nucleic acids from SARS-CoV-2, in remote regions or low resource setting without specialized equipment. Also, such methods, reagents, and devices described herein may allow for detection of target nucleic acid from SARS-CoV-2 in healthcare clinics or doctor offices without specialized equipment. In some cases, this provides a point of care testing for users to quickly and easily test for COVID-19 at home or in the office of a healthcare provider. Assays that deliver results in under an hour, for example, in 15 to 60 minutes, are particularly desirable for at home testing for many reasons. For example, antiviral medications (e.g., remdesivir), convalescent plasma, anti-SARS-CoV-2 antibodies etc., can be most effective when administered within the first 48 hours after viral exposure. Thus, the methods disclosed herein, which are capable of delivering results in under an hour, may allow for the delivery of anti-viral, anti-SARS-CoV-2 therapy during the first 48 hours after infection. Additionally, the systems and assays provided herein, which are capable of delivering quick diagnoses and results, can help keep or send a patient home, improve comprehensive disease surveillance, and prevent the spread of an infection. In other cases, this provides a test, which can be used in a lab to detect one or more nucleic acid populations or varieties of interest in a subject's sample or an environmental sample. In particular, provided herein are methods, reagents, and devices, wherein the high sensitivity lab tests can be performed in a single assay. In some cases, this may be valuable in detecting diseases in a developing country and as a global healthcare tool to detect the spread of a disease or efficacy of a treatment or provide early detection of a disease.

Some methods are described herein utilize an editing technique, such as a technique using an editing enzyme or a programmable nuclease and guide nucleic acid, to detect a target nucleic acid from SARS-CoV-2. An editing enzyme or a programmable nuclease in the editing technique can be activated by one or more target nucleic acids, after which the activated editing technique can be activated by one or more target nucleic acids, after which the activated editing enzyme or activated programmable nuclease can cleave nearby single-stranded nucleic acids, such as detector nucleic acids having a detection moiety. A target nucleic acid from SARS-CoV-2 can be amplified by RPA and then an editing technique can be used to detect the marker (e.g., amplified nucleic acid). In some instances, the editing technique can comprise an editing enzyme or programmable nuclease that, when activated, cleaves nearby RNA or DNA as the readout of the detection. The methods as described herein in some instances comprise obtaining a cell-free RNA sample, reverse transcribing the RNA sample to cDNA, amplifying the cDNA, and using an editing technique to cleave detector nucleic acids, and reading the output of the editing technique. In other instance, the method comprises obtaining a fluid sample from a patient, and without amplifying a nucleic acid of the fluid sample, using an editing technique to cleave detector nucleic acids, and detecting the nucleic acid. The method can also comprise using single-stranded detector DNA, cleaving the single-stranded detector DNA using an activated editing enzyme, wherein the editing enzyme cleaves at least 50% of a population of single-stranded detector DNA as measured by a change in color. A number of samples, guide nucleic acids, programmable nucleases or editing enzymes, support mediums, target nucleic acids, single-stranded detector nucleic acids, and reagents that are used with said devices, systems, fluidic devices, kits and methods disclosed herein.

Also disclosed herein are detector nucleic acids and methods detecting a target nucleic acid using the detector nucleic acids. Often, the detector nucleic acid is a protein-nucleic acid. For example, a method of assaying for a target nucleic acid from SARS-CoV-2 in a sample comprises contacting the sample to a plurality of complexes comprising a guide nucleic acid, each guide nucleic acid sequence comprising a segment that is reverse complementary to a segment of a target nucleic acid sequence from SARS-CoV-2 with a target nucleic acid population and programmable nucleases that exhibits sequence independent cleavage upon forming complexes comprising the segment of the guide nucleic acid binding to the segment of the target nucleic acid; and assaying for a signal indicating cleavage of at least some protein-nucleic acids of a population of protein-nucleic acids, wherein the signal indicates a presence of one or more of the target nucleic acid populations in the sample and wherein absence of the signal indicates an absence of the target nucleic acid population in the sample. Often, the protein-nucleic acid is an enzyme-nucleic acid or an enzyme substrate-nucleic acid. The nucleic acid can be DNA, RNA, or a DNA/RNA hybrid. The methods described herein use a programmable nuclease, such as the CRISPR/Cas system, to detect a target nucleic acid (e.g., SARS-CoV-2 RNA

A number of samples are consistent with the methods, reagents, and devices disclosed herein. These samples can comprise a target nucleic acid from SARS-CoV-2 for detection. The sample can be a raw sample of a sample that has been manipulated to purify nucleic acids. Generally, a sample from an individual or an animal, or an environmental sample can be obtained to test for the presence of SARS-CoV-2. A biological sample from the individual may be a sample selected from blood, serum, plasma, saliva, urine, mucosal, peritoneal, cerebrospinal, gastric secretions, nasal secretions, sputum, pharyngeal exudates, urethral or vaginal secretions, an exudate, an effusion, or tissue. A tissue sample may be dissociated or liquefied prior to use in the methods of the disclosure. A sample from an environment may be from soil, air, sewage or water. In some instances, the environmental sample is collected by using a swab. In a particular embodiment, the sample is unprocessed and used directly in the methods of the disclosure. In another embodiment, the sample is diluted with a buffer or a fluid or concentrated prior to application to the detection system or be applied neat to the detection system. Sometimes, less than 20 μL of the sample is used in the methods of the disclosure. The sample in some embodiments is in a volume of 1 μL, 2 μL, 3 μL, 4 μL, 5 μL, 6 μL, 7 μL, 8 μL, 9 μL, 10 μL, 11 μL, 12 μL, 13 μL, 14 μL, 15 μL, 16 μL, 17 μL, 18 μL, 19 μL, 20 μL, 25 μL, 30 μL, 35 μL, 40 μL, 45 μL, 50 μL, 55 μL, 60 μL, 65 μL, 70 μL, 75 μL, 80 μL, 85 μL, 90 μL, 95 μL, 100 μL, 200 μL, 300 μL, 400 μL, 500 μL, or a range that includes or is between any two of the foregoing values.

A number of target nucleic acids from SARS-CoV-2 can be used in the methods disclosed herein. The methods of the disclosure can detect a target nucleic acid present in the sample in various concentrations or amounts as a target nucleic acid. In some cases, the sample has at least 2 target nucleic acids. In some cases, the sample has 3, 5, 10, 20, 30, 40, 50, 60, 70, 80, 90, 100, 150, 200, 250, 300, 350, 400, 450, 500, 750, 1000, 2000, 3000, 4000, 5000, 6000, 7000, 8000, 9000, or 10,000 target nucleic acids. In some cases, the method detects target nucleic acids present at rate of one copy per 10¹ non-target nucleic acids, 10² non-target nucleic acids, 10³ non-target nucleic acids, 10⁴ non-target nucleic acids, 10¹ non-target nucleic acids, 10⁶ non-target nucleic acids, 10⁷ non-target nucleic acids, 108 non-target nucleic acids, 10⁹ non-target nucleic acids, 10¹⁰ non-target nucleic acids, or a range that includes or is between any two of the foregoing values.

A number of target nucleic acids from SARS-CoV-2 are consistent with the methods or compositions disclosed herein. In a particular embodiment, the methods of the disclosure detect two or more target nucleic acid sequences present in the sample in various concentrations or amounts. In some cases, the sample has at least 2 target nucleic acid sequences from SARS-CoV-2. In other cases, the sample has at least 3, 4, 5, 6, 7, 8, 9, 10, 20, 30, 40, 50 target nucleic acid sequences from SARS-CoV-2, or a range that includes or is between any two of the foregoing values.

Any of the above disclosed samples are consistent with systems, assays, and programmable nucleases disclosed herein and can be used as a companion diagnostic with SARS-CoV-2, or can be used in reagent kits, point-of-care diagnostics, or over-the-counter diagnostics.

A number of reagents are compatible with the methods, compositions and devices disclosed herein. The reagents described herein for detecting SARS-CoV-2 comprise multiple guide nucleic acids, each guide nucleic acid being specific to a target nucleic acid segment indicative of SARS-CoV-2. Each guide nucleic acid binds to the target SARS-CoV-2 nucleic acid comprising a segment of a nucleic acid sequence from SARS-CoV-2 as described herein. Each guide nucleic acid is complementary to a target nucleic acid. Often the guide nucleic acid binds specifically to the target nucleic acid.

Disclosed herein are methods of assaying for a plurality of target SARS-CoV-2 nucleic acids. For example, a method of assaying a plurality of target SARS-CoV-2 nucleic acids in a sample comprises contacting the sample to a complex comprising a plurality of guide nucleic acid sequences, each guide nucleic acid comprising a segment that is reverse complementary to a segment of the target SARS-CoV-2 nucleic acid, and programmable nucleases that exhibit sequence independent cleavage upon forming a complex comprising the segment of the guide nucleic acid binding to the segment of the target SARS-CoV-2 nucleic acid; and assaying for a signal indicating cleavage of at least some protein-nucleic acids of a population of protein-nucleic acids, wherein the signal indicates a presence of one or more target SARS-CoV-2 nucleic acids of the plurality of target SARS-CoV-2 nucleic acids in the sample and wherein absence of the signal indicates an absence of the target SARS-CoV-2 nucleic acids in the sample. As another example, a method of assaying for a target SARS-CoV-2 nucleic acid in a sample, for example, comprises: (a) contacting the sample to a plurality of complexes, each complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target SARS-CoV-2 nucleic acid; (b) contacting the plurality of complexes to a substrate; (c) contacting the substrate to a reagent that differentially reacts with a cleaved substrate; and (d) assaying for a signal indicating cleavage of the substrate, wherein the signal indicates a presence of the target SARS-CoV-2 nucleic acids in the sample and wherein absence of the signal indicates an absence of the target SARS-CoV-2 nucleic acids in the sample. Often, the substrate is an enzyme-nucleic acid. In a certain embodiment, the substrate is an enzyme substrate-nucleic acid. The disclosure further contemplates amplifying target nucleic acid in the sample prior to contacting the sample, with the plurality of complexes. The target nucleic acid can be amplified using a recombinase polymerase amplification system (RPA). RPA reactions employ recombinases which are capable of pairing sequence-specific primers with homologous sequence in duplex DNA. If target DNA is present, DNA amplification is initiated and no other sample manipulation such as thermal cycling or chemical melting is required. The entire RPA amplification system is stable as a dried formulation and can be transported safely without refrigeration. RPA reactions may also be carried out at isothermal temperatures with an optimum reaction temperature of 37-42° C. The sequence specific primers are designed to amplify a sequence comprising the target nucleic acid sequence to be detected. In certain example embodiments, a RNA polymerase promoter, such as a T7 promoter, is added to one of the primers. This results in an amplified double-stranded DNA product comprising the target sequence and a RNA polymerase promoter. After, or during, the RPA reaction, a RNA polymerase is added that will produce RNA from the double-stranded DNA templates. The amplified target RNA can then in turn be detected by a programmable nuclease systems (e.g., CRISPR Cas system) as described. In this way target DNA can be detected using the embodiments disclosed herein. RPA reactions can also be used to amplify target RNA. The target RNA is first converted to cDNA using a reverse transcriptase, followed by second strand DNA synthesis, at which point the RPA reaction proceeds as outlined above. In certain embodiments, the RPA reaction is an RT-RPA.

A programmable nuclease is a nuclease that is capable of being activated when complexed with a guide nucleic acid and target SARS-CoV-2 nucleic acid. The programmable nuclease can become activated after binding of a guide nucleic acid with a target nucleic acid, in which the activated programmable nuclease can cleave the target nucleic acid and can have trans cleavage activity. Trans cleavage activity can be non-specific cleavage of nearby single-stranded nucleic acids by the activated programmable nuclease, such as trans cleavage of detector nucleic acids with a detection moiety. Once the detector nucleic acid is cleaved by the activated programmable nuclease, the detection moiety can be released from the detector nucleic acid and generate a signal. A signal can be a calorimetric, a potentiometric, an amperometric, an optical (e.g., fluorescent, colorimetric, etc.), or a piezo-electric signal. Often, the signal is present prior to detector nucleic acid cleavage and changes upon detector nucleic acid cleavage. Sometimes, the signal is absent prior to detector nucleic acid cleavage and is present upon detector nucleic acid cleavage. The detectable signal can be immobilized on a support medium for detection. The programmable nuclease can be a CRISPR-Cas (clustered regularly interspaced short palindromic repeats—CRISPR associated) nucleoprotein complex with trans cleavage activity, which can be activated by binding of a guide nucleic acid with a target SARS-CoV-2 nucleic acid. The CRISPR-Cas nucleoprotein complex can comprise a Cas protein (also referred to as a Cas nuclease) complexed with a guide nucleic acid, which can also be referred to as CRISPR enzyme. A guide nucleic acid can be a CRISPR RNA (crRNA). Sometimes, a guide nucleic acid comprises a crRNA and a trans-activating crRNA (tracrRNA).

Several programmable nucleases are consistent with the methods and devices of the disclosure. For example, CRISPR/Cas enzymes are programmable nucleases used in the methods and systems disclosed herein. CRISPR/Cas enzymes can include any of the known Classes and Types of CRISPR/Cas enzymes. Programmable nucleases disclosed herein include Class 1 CRISPR/Cas enzymes, such as the Type I, Type III, or Type IV CRISPR/Cas enzymes. Programmable nucleases disclosed herein also include the Class 2 CRISPR/Cas enzymes, such as the Type II, Type V, and Type VI CRISPR/Cas enzymes. Typical programmable nucleases included in the several assays disclosed herein (e.g., for assaying for coronavirus in a device, such as a microfluidic device or a lateral flow assay) and methods of use thereof include a Type V or Type VI CRISPR/Cas enzyme.

In some embodiments, the Type V CRISPR/Cas enzyme is a programmable Cas12 nuclease. Type V CRISPR/Cas enzymes (e.g., Cas12 or Cas14) lack an HNH domain. A Cas12 nuclease disclosed herein cleaves nucleic acids via a single catalytic RuvC domain. The RuvC domain is within a nuclease, or “NUC” lobe of the protein, and the Cas12 nucleases further comprise a recognition, or “REC” lobe. The REC and NUC lobes are connected by a bridge helix and the Cas12 proteins additionally include two domains for PAM recognition termed the PAM interacting (PI) domain and the wedge (WED) domain. A programmable Cas12 nuclease can be a Cas12a (also referred to as Cpfl) protein, a Cas12b protein, Cas12c protein, Cas12d protein, or a Cas12e protein. In some cases, a suitable Cas12 protein comprises an amino acid sequence having at least 80%, at least 85%, at least 90%, at least 95%, at least 98%, at least 99%, or 100%, amino acid sequence identity to any one of SEQ ID NO:1-3 or 4, or to any one of accession number: ATB19153.1, WP_021736722.1, 5B43_A, 5KK5_A, 5XH7_A, 5XH6_A, WP_120110811.1, WP_093729503.1, WP_075579848.1, NLM08782.1, WP 120110807.1, WP 037975888.1, WP 081839471.1, WP_073043853.1, WP_031492824.1, WP_078933213.1, WP_048112740.1, WP_119227726.1, HAW84277.1, KF067989.1, WP_044110123.1, KIE18657.1, OFY19591.1, HHV41458.1, HHU53715.1, WP_167604087.1, WP_134744521.1, WP_167554110.1, WP_134711643.1, NDP22346.1, PKP47250.1, WP 003040289.1, ATB19155.1, WP 014550095.1, WP 003034647.1, SCH45297.1, 5NG6_A, WP_104928540.1, 6I1L_A, WP_117448340.1, 6GTC_A, 5MGA_A, WP_071304624.1, OHB41002.1, WP_137013028.1, 5NFV_A, WP_039871282.1, EFI70750.1, HHU98002.1, WP_004339290.1, WP_118080156.1, KAF3979590.1, WP_104505765.1, WP_077541740.1, OQB16057.1, SelWP 089081092.1, NLA83350.1, PIN76207.1, WP_016301126.1, WP_078273923.1, WP_112742561.1, EFI15981.1, WP_118231964.1, WP_157236615.1, WP_115369192.1, WP_014085038.1, WP_117970347.1, WP_112132723.1, WP_065256572.1, HAG50355.1, HGP76849.1, WP_118025820.1, WP_097554884.1, WP_068647445.1, WP_122892441.1, WP_015504779.1, WP_115099143.1, WP_118379198.1, WP_112744621.1, WP_045971446.1, WP_055225123.1, WP_118371518.1, WP_020988726.1, WP_117689699.1, SWP 117996653.1, WP_117685196.1, SOLA16049.1, WP_087408205.1, SWP_055272206.1, SeHAQ63770.1, WP_154266439.1, WP_118190996.1, WP_055237260.1, HCF15406.1, SWP_117714171.1, GF153642.1, WP_117918179.1, WP_118163031.1, OLA30477.1, and WP_148871677. Table 1 provide exemplary Cas12 polypeptide sequences.

TABLE 1
Cas12 Polypeptide sequences

| SEQ ID NO: | Description | Sequence |
|---|---|---|
| 1 | Acidaminococcus | MTQFEGFTNLYQVSKTLRFELIPQGKTLKHIQEQGFIEEDKARN |
| | sp. (strain | DHYKELKPIIDRIYKTYADQCLQLVQLDWENLSAAIDSYRKEKT |
| | BV3L6) | EETRNALIEEQATYRNAIHDYFIGRTDNLTDAINKRHAEIYKGL |
| | Entry NO: | FKAELFNGKVLKQLGTVTTTEHENALLRSFDKFTTYFSGFYENR |
| | U2UMQ6 | KNVFSAEDISTAIPHRIVQDNFPKFKENCHIFTRLITAVPSLRE |
| | | HFENVKKAIGIFVSTSIEEVFSFPFYNQLLTQTQIDLYNQLLGG |
| | | ISREAGTEKIKGLNEVLNLAIQKNDETAHIIASLPHRFIPLFKQ |
| | | ILSDRNTLSFILEEFKSDEEVIQSFCKYKTLLRNENVLETAEAL |
| | | FNELNSIDLTHIFISHKKLETISSALCDHWDTLRNALYERRISE |
| | | LTGKITKSAKEKVQRSLKHEDINLQEIISAAGKELSEAFKQKTS |
| | | EILSHAHAALDQPLPTTLKKQEEKEILKSQLDSLLGLYHLLDWF |
| | | AVDESNEVDPEFSARLTGIKLEMEPSLSFYNKARNYATKKPYSV |
| | | EKFKLNFQMPTLASGWDVNKEKNNGAILFVKNGLYYLGIMPKQK |
| | | GRYKALSFEPTEKTSEGFDKMYYDYFPDAAKMIPKCSTQLKAVT |
| | | AHFQTHTTPILLSNNFIEPLEITKEIYDLNNPEKEPKKFQTAYA |
| | | KKTGDQKGYREALCKWIDFTRDFLSKYTKTTSIDLSSLRPSSQY |
| | | KDLGEYYAELNPLLYHISFQRIAEKEIMDAVETGKLYLFQTYNK |
| | | DFAKGHHGKPNLHTLYWTGLFSPENLAKTSIKLNGQAELFYRPK |
| | | SRMKRMAHRLGEKMLNKKLKDQKTPIPDTLYQELYDYVNHRLSH |
| | | DLSDEARALLPNVITKEVSHEIIKDRRFTSDKFFFHVPITLNYQ |
| | | AANSPSKFNQRVNAYLKEHPETPIIGIDRGERNLIYITVIDSTG |
| | | KILEQRSLNTIQQFDYQKKLDNREKERVAARQAWSVVGTIKDLK |
| | | QGYLSQVIHEIVDLMIHYQAVVVLENLNFGFKSKRTGIAEKAVY |
| | | QQFEKMLIDKLNCLVLKDYPAEKVGGVLNPYQLTDQFTSFAKMG |
| | | TQSGFLFYVPAPYTSKIDPLTGFVDPFVWKTIKNHESRKHFLEG |
| | | FDFLHYDVKTGDFILHFKMNRNLSFQRGLPGFMPAWDIVFEKNE |
| | | TQFDAKGTPFIAGKRIVPVIENHRFTGRYRDLYPANELIALLEE |
| | | KGIVFRDGSNILPKLLENDDSHAIDTMVALIRSVLQMRNSNAAT |
| | | GEDYINSPVRDLNGVCFDSRFQNPEWPMDADANGAYHIALKGQL |
| | | LLNHLKESKDLKLQNGISNQDWLAYIQELRN |
| 2 | Francisella | MSIYQEFVNKYSLSKTLRFELIPQGKTLENIKARGLILDDEKRA |
| | tularensis | KDYKKAKQIIDKYHQFFIEEILSSVCISEDLLQNYSDVYFKLKK |
| | subsp. novicida | SDDDNLQKDFKSAKDTIKKQISEYIKDSEKFKNLFNQNLIDAKK |
| | (strain U112) | GQESDLILWLKQSKDNGIELFKANSDITDIDEALEIIKSFKGWT |
| | Entry NO: | TYFKGFHENRKNVYSSNDIPTSIIYRIVDDNLPKFLENKAKYES |
| | A0Q7Q2 | LKDKAPEAINYEQIKKDLAEELTFDIDYKTSEVNQRVFSLDEVF |
| | | EIANFNNYLNQSGITKFNTIIGGKFVNGENTKRKGINEYINLYS |
| | | QQINDKTLKKYKMSVLFKQILSDTESKSFVIDKLEDDSDVVTTM |
| | | QSFYEQIAAFKTVEEKSIKETLSLLFDDLKAQKLDLSKIYFKND |
| | | KSLTDLSQQVFDDYSVIGTAVLEYITQQIAPKNLDNPSKKEQEL |
| | | IAKKTEKAKYLSLETIKLALEEFNKHRDIDKQCRFEEILANFAA |
| | | IPMIFDEIAQNKDNLAQISIKYQNQGKKDLLQASAEDDVKAIKD |
| | | LLDQTNNLLHKLKIFHISQSEDKANILDKDEHFYLVFEECYFEL |
| | | ANIVPLYNKIRNYITQKPYSDEKFKLNFENSTLANGWDKNKEPD |
| | | NTAILFIKDDKYYLGVMNKKNNKIFDDKAIKENKGEGYKKIVYK |
| | | LLPGANKMLPKVFFSAKSIKFYNPSEDILRIRNHSTHTKNGSPQ |
| | | KGYEKFEFNIEDCRKFIDFYKQSISKHPEWKDFGFRFSDTQRYN |
| | | SIDEFYREVENQGYKLTFENISESYIDSVVNQGKLYLFQIYNKD |
| | | FSAYSKGRPNLHTLYWKALFDERNLQDVVYKLNGEAELFYRKQS |
| | | IPKKITHPAKEAIANKNKDNPKKESVFEYDLIKDKRFTEDKFFF |
| | | HCPITINFKSSGANKFNDEINLLLKEKANDVHILSIDRGERHLA |
| | | YYTLVDGKGNIIKQDTFNIIGNDRMKTNYHDKLAAIEKDRDSAR |
| | | KDWKKINNIKEMKEGYLSQVVHEIAKLVIEYNAIVVFEDLNFGF |
| | | KRGRFKVEKQVYQKLEKMLIEKLNYLVFKDNEFDKTGGVLRAYQ |
| | | LTAPFETFKKMGKQTGIIYYVPAGFTSKICPVTGFVNQLYPKYE |
| | | SVSKSQEFFSKFDKICYNLDKGYFEFSFDYKNFGDKAAKGKWTI |
| | | ASFGSRLINFRNSDKNHNWDTREVYPTKELEKLLKDYSIEYGHG |
| | | ECIKAAICGESDKKFFAKLTSVLNTILQMRNSKTGTELDYLISP |
| | | VADVNGNFFDSRQAPKNMPQDADANGAYHIGLKGLMLLGRIKNN |
| | | QEGKKLNLVIKNEEYFEFVQNRNN |
| 3 | Alicyclobacillus | MAVKSIKVKLRLDDMPEIRAGLWKLHKEVNAGVRYYTEWLSLLR |
| | acidoterrestris | QENLYRRSPNGDGEQECDKTAEECKAELLERLRARQVENGHRGP |
| | (strain ATCC | AGSDDELLQLARQLYELLVPQAIGAKGDAQQIARKFLSPLADKD |
| | 49025 | AVGGLGIAKAGNKPRWVRMREAGEPGWEEEKEKAETRKSADRTA |
| | | DVLRALADFGLKPLMRVYTDSEMSSVEWKPLRKGQAVRTWDRDM |
| | | FQQAIERMMSWESWNQRVGQEYAKLVEQKNRFEQKNFVGQEHLV |
| | | HLVNQLQQDMKEASPGLESKEQTAHYVTGRALRGSDKVFEKWGK |
| | | LAPDAPFDLYDAEIKNVQRRNTRRFGSHDLFAKLAEPEYQALWR |
| | | EDASFLTRYAVYNSILRKLNHAKMFATFTLPDATAHPIWTRFDK |
| | | LGGNLHQYTFLFNEFGERRHAIRFHKLLKVENGVAREVDDVTVP |
| | | ISMSEQLDNLLPRDPNEPIALYFRDYGAEQHFTGEFGGAKIQCR |
| | | RDQLAHMHRRRGARDVYLNVSVRVQSQSEARGERRPPYAAVFRL |
| | | VGDNHRAFVHFDKLSDYLAEHPDDGKLGSEGLLSGLRVMSVDLG |
| | | LRTSASISVFRVARKDELKPNSKGRVPFFFPIKGNDNLVAVHER |
| | | SQLLKLPGETESKDLRAIREERQRTLRQLRTQLAYLRLLVRCGS |
| | | EDVGRRERSWAKLIEQPVDAANHMTPDWREAFENELQKLKSLHG |
| | | ICSDKEWMDAVYESVRRVWRHMGKQVRDWRKDVRSGERPKIRGY |
| | | AKDVVGGNSIEQIEYLERQYKFLKSWSFFGKVSGQVIRAEKGSR |
| | | FAITLREHIDHAKEDRLKKLADRIIMEALGYVYALDERGKGKWV |
| | | AKYPPCQLILLEELSEYQFNNDRPPSENNQLMQWSHRGVFQELI |
| | | NQAQVHDLLVGTMYAAFSSRFDARTGAPGIRCRRVPARCTQEHN |
| | | PEPFPWWLNKFVVEHTLDACPLRADDLIPTGEGEIFVSPFSAEE |
| | | GDFHQIHADLNAAQNLQQRLWSDFDISQIRLRCDWGEVDGELVL |
| | | IPRLTGKRTADSYSNKVFYTNTGVTYYERERGKKRRKVFAQEKL |
| | | SEEEAELLVEADEAREKSVVLMRDPSGIINRGNWTRQKEFWSMV |
| | | NQRIEGYLVKQIRSRVPLQDSACENTGDI |
| 4 | Lachnospiraceae | MSKLEKFTNCYSLSKTLRFKAIPVGKTQENIDNKRLLVEDEKRA |
| | bacterium | EDYKGVKKLLDRYYLSFINDVLHSIKLKNLNNYISLFRKKTRTE |
| | Entry No. | KENKELENLEINLRKEIAKAFKGNEGYKSLFKKDIIETILPEFL |
| | A0A5S8WF58 | DDKDEIALVNSFNGFTTAFTGFFDNRENMFSEEAKSTSIAFRCI |
| | | NENLTRYISNMDIFEKVDAIFDKHEVQEIKEKILNSDYDVEDFF |
| | | EGEFFNFVLTQEGIDVYNAIIGGFVTESGEKIKGLNEYINLYNQ |
| | | KTKQKLPKFKPLYKQVLSDRESLSFYGEGYTSDEEVLEVFRNTL |
| | | NKNSEIFSSIKKLEKLFKNFDEYSSAGIFVKNGPAISTISKDIF |
| | | GEWNVIRDKWNAEYDDIHLKKKAVVTEKYEDDRRKSFKKIGSFS |
| | | LEQLQEYADADLSVVEKLKEIIIQKVDEIYKVYGSSEKLFDADF |
| | | VLEKSLKKNDAVVAIMKDLLDSVKSFENYIKAFFGEGKETNRDE |
| | | SFYGDFVLAYDILLKVDHIYDAIRNYVTQKPYSKDKFKLYFQNP |
| | | QFMGGWDKDKETDYRATILRYGSKYYLAIMDKKYAKCLQKIDKD |
| | | DVNGNYEKINYKLLPGPNKMLPKVFFSKKWMAYYNPSEDIQKIY |
| | | KNGTFKKGDMFNLNDCHKLIDFFKDSISRYPKWSNAYDFNFSET |
| | | EKYKDIAGFYREVEEQGYKVSFESASKKEVDKLVEEGKLYMFQI |
| | | YNKDFSDKSHGTPNLHTMYFKLLFDENNHGQIRLSGGAELFMRR |
| | | ASLKKEELVVHPANSPIANKNPDNPKKTTTLSYDVYKDKRFSED |
| | | QYELHIPIAINKCPKNIFKINTEVRVLLKHDDNPYVIGIDRGER |
| | | NLLYIVVVDGKGNIVEQYSLNEIINNFNGIRIKTDYHSLLDKKE |
| | | KERFEARQNWTSIENIKELKAGYISQVVHKICELVEKYDAVIAL |
| | | EDLNSGFKNSRVKVEKQVYQKFEKMLIDKLNYMVDKKSNPCATG |
| | | GALKGYQITNKFESFKSMSTQNGFIFYIPAWLTSKIDPSTGFVN |
| | | LLKTKYTSIADSKKFISSFDRIMYVPEEDLFEFALDYKNFSRTD |
| | | ADYIKKWKLYSYGNRIRIFRNPKKNNVFDWEEVCLTSAYKELFN |
| | | KYGINYQQGDIRALLCEQSDKAFYSSFMALMSLMLQMRNSITGR |
| | | TDVDFLISPVKNSDGIFYDSRNYEAQENAILPKNADANGAYNIA |
| | | RKVLWAIGQFKKAEDEKLDKVKIAISNKEWLEYAQTSVKH |

Alternatively, the type V CRISPR/Cas enzyme is a programmable Cas14 nuclease. A Cas14 protein of the disclosure include 3 partial RuvC domains (RuvC-I, RuvC-II, and RuvC-III, also referred herein as subdomains) that are not contiguous with respect to the primary amino acid sequence of the Cas14 protein, but form a RuvC domain once the protein is produced and folded. A naturally occurring Cas14 protein functions as an endonuclease that catalyzes cleavage at a specific sequence in a target nucleic acid. A programmable Cas14 nuclease can be a Cas14a protein, a Cas14b protein, a Cas14c protein, a Cas14d protein, a Cas14e protein, a Cas14f protein, a Cas14g protein, a Cas14h protein, or a Cas14u protein. In some cases, a suitable Cas14 protein comprises an amino acid sequence having at least 80%, at least 85%, at least 90%, at least 95%, at least 98%, at least 99% identity to any one of Accession numbers or WP_048402777.1, WP_077210027.1, WP_083314917.1, WP_018297204.1, WP_014302663.1, WP_088264504.1, CAB0532088.1, CAB0624945.1, CAB0539033.1, CAB1026072.1, WP_014309766.1, CAB0670887.1, CAB0991295.1, WP_014306233.1, WP_082258207.1, CAB0668382.1, WP_014307728.1, WP_071570672.1, CAB0674275.1, CAB0772394.1, WP_016830429.1, CAB0715588.1, CAB0625127.1, WP_014309003.1, CAB1027305.1, WP_088267460.1, WP_072588167.1, CAB0529362.1, WP_018024637.1, WP_080754080.1, WP_052251263.1, WP_082345415.1, VH000656.1, WP_082346565.1, WP_126416583.1, SU087902.1, WP_115324474.1, WP_051952360.1, WP_083290301.1, WP_041729458.1, WP_025296977.1, WP_114949752.1, WP_164978155.1, OQD32504.1, WP_070565815.1, WP_101678534.1, WP_024963699.1, WP_158381790.1, WP_126714927.1, WP_143335301.1, WP_167616985.1, WP_099981414.1, WP_070361639.1, WP_108252736.1, WP_070840746.1, WP_101733443.1, WP_071565490.1, WP_083330183.1, SDE63285.1, WP_070562523.1, WP_049620073.1, KMY22947.1, WP_070529210.1, WP_080975047.1, WP_158396338.1, WP_042531761.1, WP_165002380.1, WP_151549778.1, WP_082121333.1, WP_083317802.1, WP_157034441.1, WP_144779956.1, WP_058237269.1, WP_075664776.1, WP_115685609.1, WP_150386218.1, WP_015382092.1, WP_103759126.1, WP_070791257.1, WP_087117126.1, WP_034986778.1, WP_070551100.1, WP_035011874.1, NNO11674.1, WP_092150518.1, SeWP_071056232.1, WP_070761730.1, WP_012361195.1, WP_095537844.1, WP_034966247.1, WP_111725437.1, WP_034665223.1, WP_048378674.1, WP_095555610.1, WP_051483496.1, WP_122086339.1, WP_035007949.1, WP_126843983.1, WP_034980529.1, and VDG62294.1.

In some embodiments, the Type VI CRISPR/Cas enzyme is a programmable Cas13 nuclease. The general architecture of a Cas13 protein includes an N-terminal domain and two HEPN (higher eukaryotes and prokaryotes nucleotide-binding) domains separated by two helical domains. The HEPN domains each comprise aR-X₄-H motif. Shared features across Cas13 proteins include that upon binding of the crRNA of the guide nucleic acid to a target nucleic acid, the protein undergoes a conformational change to bring together the HEPN domains and form a catalytically active RNase. Thus, two activatable HEPN domains are characteristic of a programmable Cas13 nuclease of the present disclosure. However, programmable Cas13 nucleases also consistent with the present disclosure include Cas13 nucleases comprising mutations in the HEPN domain that enhance the CAS13 proteins cleavage efficiency or mutations that catalytically inactivate the HEPN domains.

A programmable Cas13 nuclease can be a Cas13a protein (also referred to as “c2c2”), a Cas13b protein, a Cas13c protein, a Cas13d protein, or a Cas13e protein. In some cases, a subject C2c2 protein includes an amino acid sequence having at least 80%, at least 85%, at least 90%, at least 95%, at least 97%, at least 98%, at least 99% sequence identity to an amino acid sequence as set forth in any one of accession number: WP_012985477.1, WP_099225408.1, WP_118907415.1, WP_036091002.1, WP_036059185.1, WP_013443710.1, WP_034560163.1, WP_034563842.1, WP_071146234.1, WP_149023847.1, WP_092118911.1, HCK24590.1, WP_149023846.1, WP_021747205.1, WP_015770004.1, WP_071125398.1, WP_021746774.1, WP_146998208.1, WP_146959607.1, WP_149023848.1, QID24124.1, 5XWY_A, ERL25782.1, 5XWP_A, WP_021746003.1, WP_071124126.1, BBM48975.1, NLU52303.1, NLU32351.1, OQX30025.1, HHT89324.1, WP_092122836.1, NMA74573.1, WP_132694182.1, PJI41863.1, SUS05702.1, WP_076398593.1, WP_163266467.1, WP_150214158.1, KAA6404670.1, WP_114086813.1, PIT01667.1, WP_100176879.1, WP_073955355.1, WP_133357912.1, KAA6204339.1, WP_103203632.1, WP_036059184.1, WP_013067728.1, WP_023911507.1, WP_146746344.1, RAP39618.1, WP_149023845.1, WP_150215210.1, WP_137134457.1, WP_080615427.1, WP_108028905.1, WP_031473346.1, KAA6204514.1, and WP_079495749.1.

In a certain embodiment, the programmable nuclease is Cas13. In a further embodiment, the Cas13 is selected from Cas13a, Cas13b, Case13c, Cas13d, and Cas13e. In another embodiment, the programmable nuclease can be Mad7 or Mad2. In yet another embodiment, the programmable nuclease is Cas12. In yet a further embodiment, the Cas12 is selected from Cas12a, Cas12b, Case12c, Cas12d, and Cas12e. In a certain embodiment, the programmable nuclease is selected from Csm1, Cas9, C2c4, C2c8, C2c5, C2c10, C2c9, or CasZ. In another embodiment, the Csm1 can be smCmsl, miCmsl, obCmsl, or suCmsl. In yet another embodiment, Cas13a is C2c2. In a further embodiment, CasZ is Cas14a, Cas14b, Cas14c, Cas14d, Cas14e, Cas14f, Cas14g, Cas14h, Cas14i, Cas14j, or Cas14k. In yet another embodiment, the programmable nuclease is a type V CRISPR-Cas system. In a further embodiment, the programmable nuclease is a type VI CRISPR-Cas system. In another embodiment, the programmable nuclease is a type III CRISPR-Cas system. In yet another embodiment, the programmable nuclease originated from Leptotrichia shahii, Listeria selligeri, Leptotrichia buccalis, Leptotrichia waden, Rhodobacter capsulatus, Herbinix hemicellulosilytica, Paludibacter propionicigenes, Lachnospiraceae bacterium, Eubacterium rectale, Listeria new yorkensis, Clostridium aminophilum, Prevotella sp., Capnocytophaga canimorsus, Lachnospiraceae bacterium, Bergeyella zoohelcum, Prevotella intermedia, Prevotella buccae, Alistipes sp., Riemerella anatipestifer, Prevotella aurantiaca, Prevotella saccharolytica, Prevotella intermedia, Capnocytophaga canimorsus, Porphyromonas gulae, Prevotella sp., Porphyromonas gingivalis, Prevotella intermedia, Enterococcus italicus, Lactobacillus salivarius, or Thermus thermophilus. In a further embodiment, the Cas13 is selected from LbuCas13a, LwaCas13a, LbaCas13a, HheCas13a, PprCas13a, EreCas13a, CamCas13a, and LshCas13a. The trans cleavage activity of the CRISPR enzyme can be activated when the crRNA is complexed with the target nucleic acid. The trans cleavage activity of the CRISPR enzyme can be activated when the guide nucleic acid comprising a tracrRNA and crRNA are complexed with the target SARS-CoV-2 nucleic acid.

In a particular embodiment, the disclosure provides for the use of primers for the methods disclosed herein to detect SARS-CoV-2 in a sample. Exemplary primers are provided in Table 2.

TABLE 2
Primers for SARS-CoV-2 CAS12 Assay

| PCR | Target | Sequence (SEQ ID NO :) |
|---|---|---|
| 2019-nCoV_N2-F | N gene | TTACAAACATTGGCCGCAAA (SEQ ID NO: 12) |
| 2019-nCoV_N2-R | N gene | GCGCGACATTCCGAAGAA (SEQ ID NO: 13) |
| 2019-nCoV_N2-Probe | N gene | /56-FAM/ACAATTTGCCCCCAGCGCTTCAG/ |
| | | 3BHQ_1/ (SEQ ID NO: 14) |
| RP-F_Tm55.9 | Human RNase P | CGAGGTGGGACTTCAGCATG (SEQ ID NO: 15) |
| RP-R_Tm55.9 | Human RNase P | GAGCGGCTGTCTCCACAAGT (SEQ ID NO: 16) |
| RP-Probe | Human RNase P | /56-FAM/TTCTGACCTGAAGGCTCTGCGCG/ |
| | | 3BHQ_1/ (SEQ ID NO: 17) |
| E-Sarbeco_F1 | Human RNase P | ACAGGTACGTTAATAGTTAATAGCGT (SEQ ID |
| | | NO: 18) |
| E-Sarbeco_R2 | E gene | ATATTGCAGCAGTACGCACACA (SEQ ID NO: 19) |
| E-Sarbeco | E gene | /56-FAM/ACACTAGCCATCCTTACTGCGCTTCG |
| Probe | | /3BHQ1/ (SEQ ID NO: 20) |
| RPA primers | Target | Sequence |
| E- | E gene | GAAGAGACAGGTACGTTAATAGTTAATAGCGT (SEQ |
| Sarbeco_R1_longer_ | | ID NO: 5) |
| Tm59.3_32nt | | |
| E-Sarbeco- | E gene | ACGTTAACAATATTGCAGCAGTACGCACACA (SEQ |
| R2_longer | | ID NO: 21) |
| Tm60.4_31nt | | |
| 2019-nCoV_N2- | N gene | ACAAGGAACTGATTACAAACATTGGCCGCAAA (SEQ |
| F_longer_Tm60.5_32 | | ID NO: 7) |
| nt | | |
| 2019-nCoV_N2- | N gene | TTCCATGCCAATGCGCGACATTCCGAAGAA (SEQ ID |
| R_longer_Tm63_30nt | | NO: 22) |
| RP-F_mod_longer_ | Human RNase P | GAATTCGGCACGAGGTGGGACTTCAGCATG (SEQ ID |
| Tm65.7_32-bp | | NO: 23) |
| RP- | Human RNase P | AATAGCCAAGGTGAGCGGCTGTCTCCACAAGT (SEQ |
| R_longer_Tm65.7_32 | | ID NO: 24) |
| bp | | |
| RPA nested primers | Target | Sequence |
| E- | E gene | GGACCAGGAACTAATCAGACAAGGAACTGA (SEQ ID |
| Sarbeco_R1_longer2_ | | NO: 25) |
| Tm61.6_30nt | | |
| E- | E gene | AGGTCAACCACGTTCCCGAAGGTGTGACTT (SEQ ID |
| Sarbeco_R2_longer2_ | | NO: 26) |
| Tm64.4_30nt | | |
| 2019-ncov_N2- | N gene | GGCAGACGUGGUCCAGAACAAACCCAAGGA (SEQ ID |
| F_longer2_ | | NO: 27) |
| Tm66_31nt | | |
| 2019-nCoV_N2- | N gene | TAGGTCAACCACGTTCCCGAAGGTGTGACTT (SEQ |
| R_longer2_ | | ID NO: 28) |
| Tm64_31nt | | |
| Oligonucleotide | | |
| Templates | | |
| Rnase-P-oligo- | | TAATACGACTCACTATAGGAATTCGGCACGAGGTG |
| 178bp | | GGACTTCAGCATGGCGGTGTTTGCAGATTTGGACC |
| | | TGCGAGCGGGTTCTGACCTGAAGGCTCTGCGCGGA |
| | | CTTGTGGAGACAGCCGCTCACCTTGGCTATTCAGT |
| | | TGTTGCTATCAATCATATCGTTGACTTTAAGGAAA |
| | | AGAAAC (SEQ ID NO: 29) |
| N2-oligo-181bp | | TAATACGACTCACTATAGAACAAACCCAAGGAAAT |
| | | TTTGGGGACCAGGAACTAATCAGACAAGGAACTGA |
| | | TTACAAACATTGGCCGCAAATTGCACAATTTGCCC |
| | | CCAGCGCTTCAGCGTTCTTCGGAATGTCGCGCATT |
| | | GGCATGGAAGTCACACCTTCGGGAACGTGGTTGAC |
| | | CTACACAGGTG (SEQ ID NO: 30) |

Described herein are reagents comprising a detection moiety, wherein the detector nucleic acid is capable of being cleaved by the activated nuclease, thereby generating a first detectable signal. As used herein, a detector nucleic acid is used interchangeably with report or reporter molecules. In some embodiments, the detector nucleic acid is a single-stranded nucleic acid comprising deoxyribonucleotides. In other cases, the detector nucleic acid is a single-stranded nucleic acid comprising ribonucleotides. The detector nucleic acid can be a single-stranded nucleic acid comprising at least one deoxyribonucleotide and at least one ribonucleotide. In other embodiments, the detector nucleic acid is a single-stranded nucleic acid comprising at least one ribonucleotide residue at an internal position that functions as a cleavage site. In some embodiments, the detector nucleic acid comprises at least 2, 3, 4, 5, 6, 7, 8, 9 or 10 ribonucleotide residues at an internal position. Sometimes the ribonucleotide residues are continuous. Alternatively, the ribonucleotide residues are interspersed in between non-ribonucleotide residues. In some embodiments, the detector nucleic acid has only ribonucleotide residues. In other embodiments, the detector nucleic acid has only deoxyribonucleotide residues. In some embodiments, the detector nucleic acid is 5, 6, 7, 8, 9, 10, 11, 12, 13, 14, 15, 16, 17, 18, 19, 20, 21, 22, 23, 24, or 25 nucleotides in length, or is range that includes or is between any two of the foregoing lengths.

The single-stranded detector nucleic acid comprises a detection moiety capable of generating a first detectable signal. In a certain embodiment, the detector nucleic acid comprises a protein capable of generating a signal. A signal can be a calorimetric, potentiometric, amperometric, optical (e.g., fluorescent, luminescent, etc.), or piezo-electric. In some embodiments, a detection moiety is located on one side of the detector nucleic acid cleavage site. In further embodiments, a quenching moiety is on the other side of the cleavage site. In some embodiments, the quenching moiety is 5′ to the cleavage site and the detection moiety is 3′ to the cleavage site. In other embodiments, the detection moiety is 5′ to the cleavage site and the quenching moiety is 3′ to the cleavage site. In further embodiments, the quenching moiety is at the 5′ terminus of the detector nucleic acid. In alternate embodiments, the quenching moiety is at the 3′ terminus of the detector nucleic acid. In further embodiments, the detection moiety is at the 5′ terminus of the detector nucleic acid. In alternate embodiments, the detection moiety is at the 3′ terminus of the detector nucleic acid.

A detection moiety can be an infrared fluorophore. A detection moiety can be a fluorophore that emits fluorescence at 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, or a range that includes or is between any two of the foregoing wavelengths. A detection moiety can be a fluorophore that emits a fluorescence in the same range as fluorescein, 6-fluoresceine, IRDYE 700, TYE 665, Alexa Fluor, or ATTO TM633. A detection moiety can be fluorescein, 6-fluoresceine, IRDYE 700, TYE 665, Alexa Fluor, or ATTO TM633.

A quenching moiety can be chosen based on its ability to quench the detection moiety. A quenching moiety can quench the fluorescence emitted by a fluorophore at 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, or a range that includes or is between any two of the foregoing wavelengths. A quenching moiety can quench fluorescence emitted by fluorescein, 6-fluoresceine, IRDYE 700, TYE 665, Alexa Fluor, or ATTO TM633. A detection moiety can be fluorescein, 6-fluoresceine, IRDYE 700, TYE 665, Alexa Fluor, or ATTO TM633. A quenching moiety can be Iowa Black RQ, Iowa Black FQ, and Black Hole quencher.

The generation of the detectable signal from the release of the detection moiety indicates that cleavage by the programmable nuclease has occurred and that the sample contains the target nucleic acid. In some embodiments, the detection moiety comprises a fluorescent dye. In a certain embodiment, the detection moiety comprises a fluorescence resonance energy transfer (FRET) pair. In some embodiments, the detection moiety comprises an infrared (IR) dye. In some embodiments, the detection moiety comprises and ultraviolet (UV) dye.

A number of detection devices and methods are consistent with the methods disclosed herein. For example, any device that can measure or detect a calorimetric, potentiometric, amperometric, optical (e.g., fluorescent, luminescent, etc.), or piezo-electric signal. For optical signals, the signals can be visualized by eye (e.g., a lateral flow assay), or by use of detection device (e.g., a microplate reader, camera, etc.). In a particular embodiment, a lateral flow assay is used to detect a target nucleic acid from SARS-CoV-2 (e.g., see FIG. 2).

Lateral flow assay (LFA) based devices are among very rapidly growing strategies for qualitative and quantitative analysis. Lateral flow assays are performed over a strip, different parts of which are assembled on a plastic backing. These parts include a sample application pad, a conjugate pad, a nitrocellulose membrane and an adsorption pad. The nitrocellulose membrane is further divided into test and control lines. Pre-immobilized reagents at different parts of the strip become active upon flow of liquid sample. Lateral flow assays combine unique advantages of biorecognition probes and chromatography. Lateral flow assays basically combine a number of variants such as formats, biorecognition molecules, labels, detection systems and application. Strips used for lateral flow assays contain four main components: a sample application pad, a conjugate pad, nitrocellulose membranes, and an adsorbent pad.

Sample application pad: The sample application pad is made of cellulose and/or glass fiber. The sample is applied on this pad to start the assay. Its function is to transport the sample to other components of lateral flow test strip (LFTS). The sample pad should be capable of transportation of the sample in a smooth, continuous and homogenous manner. The sample application pads are sometimes designed to pretreat the sample before its transportation. This pretreatment may include separation of sample components, removal of interfering agents, adjustment of pH, etc.

Conjugate pad: The conjugate pad is the place where labeled biorecognition molecules are dispensed. The material of conjugate pad should immediately release labeled conjugate upon contact with moving liquid sample. The labeled conjugate should stay stable over entire life span of the lateral flow strip. Any variations in dispensing, drying or release of conjugate can change results of the assay significantly. Poor preparation of labeled conjugate can adversely affect sensitivity of assay. Glass fiber, cellulose, poly-esters and some other materials are used to make conjugate pad for the lateral flow assay. The nature of the conjugate pad material has an effect on release of labeled conjugate and sensitivity of assay.

Nitrocellulose membrane: The Nitrocellulose membrane is highly important in determining sensitivity of the lateral flow assay. Nitrocellulose membranes are available in different grades. Test and control lines are drawn over this piece of membrane. An ideal membrane should provide support and good binding to capture probes (antibodies, aptamers etc.). Nonspecific adsorption over test and control lines may affect results of the assay significantly, thus a good membrane will be characterized by lesser non-specific adsorption in the regions of test and control lines. Wicking rate of nitrocellulose membrane can influence assay sensitivity. These membranes are easy to use, inexpensive, and offer high affinity for proteins and other biomolecules. Proper dispensing of bioreagents, drying and blocking play a role in improving sensitivity of assay.

Adsorbent pad: The adsorbent pad works as sink at the end of the strip. It also helps in maintaining the flow rate of the liquid over the membrane and stops back flow of the sample. The adsorbent capacity to hold liquid can play an important role in results of assay. All these components are fixed or mounted over a backing card.

Various formats can be adopted into the lateral flow assay, including the sandwich format, the competitive format and the multiplex detection format.

Sandwich Format: In a typical sandwich format, label (enzymes or nanoparticles or fluorescent dyes) coated antibody or aptamer is immobilized at the conjugate pad. This is a temporary adsorption which can be flushed away by flow of any buffer solution. A primary antibody or aptamer against target analyte is immobilized over test line. A secondary antibody or probe against labeled conjugate antibody/aptamer is immobilized at control zone. Sample containing the analyte is applied to the sample application pad and it subsequently migrates to the other parts of strip. At the conjugate pad, target analyte is captured by the immobilized labeled antibody or aptamer conjugate and results in the formation of labeled antibody conjugate/analyte complex. This complex now reaches the nitrocellulose membrane and moves under capillary action. At the test line, labeled antibody conjugate/analyte complex is captured by another antibody which is primary to the analyte. The analyte becomes sandwiched between the labeled and primary antibodies forming a labeled antibody conjugate/analyte/primary antibody complex. Excess labeled antibody conjugate will be captured at a control zone by a secondary antibody. Buffer or excess solution goes to absorption pad. The intensity of color at the test line corresponds to the amount of target analyte and is measured with an optical strip reader or visually inspected. Appearance of color at control line ensures that a strip is functioning properly.

Competitive format: A competitive format suits best for low molecular weight compounds which cannot bind two antibodies simultaneously. Absence of color at test line is an indication for the presence of analyte while appearance of color both at test and control lines indicates a negative result. The competitive format has two layouts. In the first layout, solution containing target analyte is applied onto the sample application pad and prefixed labeled biomolecule (antibody/aptamer) conjugate gets hydrated and starts flowing with the moving liquid. The test line contains pre-immobilized antigen (same analyte to be detected) which binds specifically to label conjugate. Control line contains pre-immobilized secondary antibody which has the ability to bind with labeled antibody conjugate. When liquid sample reaches at the test line, pre-immobilized antigen will bind to the labeled conjugate in case target analyte in sample solution is absent or present in such a low quantity that some sites of labeled antibody conjugate were vacant. Antigen in the sample solution and the one which is immobilized at test line of strip compete to bind with labeled conjugate. In another layout, labeled analyte conjugate is dispensed at conjugate pad while a primary antibody to analyte is dispensed at the test line. After application of analyte solution, a competition takes place between analyte and labeled analyte to bind with primary antibody at test line.

Multiplex detection: Multiplex detection format is used for detection of more than one target species and assay is performed over the strip containing test lines equal to number of target species to be analyzed. It is highly desirable to analyze multiple analytes simultaneously under same set of conditions. Multiplex detection format is very useful in clinical diagnosis where multiple analytes which are inter-dependent in deciding about the stage of a disease are to be detected. Lateral flow strips for this purpose can be built in various ways, i.e., by increasing length and test lines on conventional strip, making other structures like stars or T-shapes.

Various biorecognition molecules can be used with the lateral flow assay, including antibodies, aptamers, and molecular beacons.

Antibodies: Antibodies are employed as biorecognition molecules on the test and control lines of lateral flow strip and they bind to target analyte through immunochemical interactions. Resulting assay is known as lateral flow immunochromatographic assay (LFIA). Antibodies are available against common contaminants but they can also be synthesized against specific target analytes. An antibody which specifically binds to a certain target analyte is known as primary antibody but the one which is used to bind a target containing designs, formats and applications of lateral flow assay antibody or another antibody is known as secondary antibody.

Aptamers: Aptamers are the artificial nucleic acids and their discovery was reported by two groups in 1990. Aptamers have very high association constants and can bind selectively with a variety of target analytes. Organic molecules having molecular weights in the range of 100-10,000 Da are outstanding targets for aptamers. Because of their unique affinity toward target molecules, very closely related interferences can be differentiated. They are preferred over antibodies due to many features which include easy production process, simple labeling process, amplification after selection, straightforward structure modifications, unmatched stability, reproducibility and versatility of closely located quencher.

Molecular beacons: Molecular beacons can bind with high specificity and selectivity to nucleic acid sequences, toxins, proteins and other target molecules. Molecular beacons are composed of 15-30 base pairs in loop which are complimentary to target analyte and 4-6 base pairs at double stranded stem. Molecular beacons are being used in messenger RNA detection, intercellular imaging, protein and small molecule analysis, biosensors, biochip development, single nucleotide polymorphism and gene expression studies.

The list of materials that can be used as a label in a lateral flow assay is extensive and includes gold nanoparticles, colored latex beads, magnetic particles, carbon nanoparticles, selenium nanoparticles, silver nanoparticles, quantum dots, up converting phosphors, organic fluorophores, textile dyes, enzymes, liposomes and others. Any material that is used as a label should be detectable at very low concentrations and it should retain its properties upon conjugation with biorecognition molecules. This conjugation is also expected not to change the features of the bio-recognition probes. The ease in conjugation with biomolecules and stability over longer period of time are desirable features for a good label. Concentrations of labels down to 10-9M are optically detectable. After the completion of assay, some labels generate direct signals (as color from gold colloidal) while others require additional steps to produce analytical signals (as enzymes produce detectable product upon reaction with suit-able substrate). Hence the labels which give direct signal are preferable in LFA because of less time consumption and reduced procedure.

Colloidal gold nanoparticles are the most commonly used labels in LFA. Colloidal gold is inert and gives very perfect spherical particles. These particles have very high affinity toward biomolecules and can be easily functionalized. Optical properties of gold nanoparticles are dependent on size and shape. Size of particles can be tuned by use of suitable chemical additives. Their unique features include environment friendly preparation, high affinity toward proteins and biomolecules, enhanced stability, exceptionally higher values for charge transfer and good optical signaling. Optical properties of gold nanoparticle enhance sensitivity of analysis in LFA. Sensitivity is a function of molar absorption coefficient and accumulation of gold nanoparticles on target molecule. Optical signal of gold nanoparticles in colorimetric LFA can be amplified by deposition of silver, gold nanoparticles and enzymes.

Use of magnetic particles as colored labels in LFA has been reported by number of researchers. Colored magnetic particles produce color at the test line which is measured by an optical strip reader but magnetic signals coming from magnetic particles can also be used as detection signals and recorded by a magnetic assay reader. It has been reported that magnetic signals are stable for longer time compared to optical signals and they enhance sensitivity of LFA by 10 to 1000 s fold.

Fluorescent molecules are widely used in LFA as labels and the amount of fluorescence is used to quantitate the concentration of analyte in the sample. Detection of proteins is accomplished by using organic fluorophores such as rhodamine as labels in LFA. High photostability and brightness are required for LFAs.

Quantum dots are also used in LFAs. These semiconducting particles are not only water soluble but can also be easily combined with biomolecules because of closeness in dimensions. Owing to their unique optical properties, quantum dots have come up as a substitute to organic fluorescent dyes. Like gold nanoparticles QDs show size dependent optical properties and a broad spectrum of wavelengths can be monitored. Single light source is sufficient to excite quantum dots of all different sizes. QDs have high photostability and absorption coefficients. They can retain their fluorescent properties within the cells and bodies of organisms and less susceptible to metabolic degradation because of their inorganic nature.

Upconverting phosphors (UCP) are also labels which find use in LFAs. UPA labels are characterized by their excitation in infra-red region and emission in high energy visible region. Compared to other fluorescent materials, they have a unique advantage of not showing any auto fluorescence. Because of their excitation in IR regions, they do not photo degrade biomolecules. A major advantage lies in their production from easily available bulk materials. UCP particles were found to show size dependent sensitivity and specificity for detection of antibodies using LFA in sera of patients.

Enzymes are also employed as labels in LFA. But they increase one step in LFA which is application of suitable substrate after complete assay. This substrate will produce color at test and control lines as a result of enzymatic reaction. Horse-radish peroxidase labeled antibody conjugates can be used for detection of primary animal IgGs. In case of enzymes, selection of suitable enzyme substrate combination is one necessary requirement in order to get a colored product for strip reader or electroactive product for electrochemical detection. In other words, sensitivity of detection is dependent on the enzyme/substrate combination. Enhanced LFA sensitivity was observed when enzyme loaded gold nanoparticles were used as a label.

Colloidal carbon is comparatively inexpensive LFA label and its production can be easily scaled up. Because of their black color, carbon NPs can be easily detected with high sensitivity. Colloidal carbon can be functionalized with a large variety of biomolecules for detection of low and high molecular weight analytes. Carbon black nanoparticles showed very low detection limits compared to other labels. The sensitivity of LFA employing colloidal carbon is reported to be comparable with ELISA assay.

In case of gold nanoparticles or other color producing labels, qualitative or semi-quantitative analysis can be done by visual inspection of colors at test and control lines. The major advantage of visual inspection is rapid qualitative answer in “Yes” or “NO”. Such quick replies about presence of an analyte in clinical analysis have very high importance. Such tests can help doctors or other investigators to make an immediate decision, e.g., situations where test results from central labs cannot be waited for because of huge time consumption. But for quantification, optical strip readers are employed for measurement of the intensity of colors produced at test and control lines of strip. This is achieved by inserting the strips into a strip reader and intensities are recorded simultaneously by imaging software. Optical images of the strips can also be recorded with a camera and then processed by using a suitable software. Such systems use monochromatic light and wavelength of light can be adjusted to get a good contrast among test and control lines and background. Automated systems have advantages over manual imaging and processing in terms of time consumption, interpretation of results and adjustment of variables. In case of fluorescent labels, a fluorescence strip reader is used to record fluorescence intensity of test and control lines. Fluorescence brightness of a test line increases with an analyte's concentration in the sample. Magnetic strip readers and electrochemical detectors are also reported as detection systems in LFTS but they are not as common. Selection of detector is mainly determined by the label employed in analysis.

LFA strips give qualitative or semi-quantitative results which can be observed by naked eyes. Conventional LFAs are normally qualitative and give answers as a ‘yes’ or ‘no’ result. A good LFA biosensor should have the following properties: biocompatibility, high specificity, high sensitivity, rapidity of analysis, reproducibility/precision of results, wide working range of analysis, accuracy of analysis, high through-put, compactness, low cost, simplicity of operation, portability, flexibility in configuration, possibility of miniaturization, potential of mass production and on-site detection.

For use in applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise one or more SARS-CoV-2 assay components described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprise a compound with an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself, a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein. These other therapeutic agents may be used, for example, in the amounts indicated in the Physicians' Desk Reference (PDR) or as otherwise determined by one of ordinary skill in the art.

### EXAMPLES

SARS-CoV-2 target sequences. SARS-CoV-2 target sequences were designed using all available genomes available from GISAID. Briefly, viral genomes were aligned using Clustal Omega. Next, LbCas12a target sites on the SARS-CoV-2 genome were filtered against SARS-CoV, two bat-SARS-like-CoV genomes and common human coronavirus genomes. Compatible target sites were finally compared to those used in current protocols from the CDC and WHO. LAMP primers for SARS-CoV-2 were designed against regions of the N-gene and E-gene using PrimerExplorer v5 (primerexplorer.jp/e/). RNase-P POP7 primers were originally published by Curtis, et al. (2018) and a compatible gRNA was designed to function with these primer sets.

Target RNAs were generated from synthetic gene fragments of the viral genes of interest. First a PCR step was performed on the synthetic gene fragment with a forward primer that contained a T7 promoter. Next, the PCR product was used as the template for an in-vitro transcription (IVT) reaction at 37° C. for 2 hours. The IVT reaction was then treated with TURBO DNase (Thermo) for 30 minutes at 37° C., followed by a heat-denaturation step at 75° C. for 15 minutes. RNA was purified using RNA Clean and Concentrator 5 columns (Zymo Research). RNA was quantified by Nanodrop and Qubit and diluted in nuclease-free water to working concentrations.

SARS-CoV-2-detection assays were performed using RPA for pre-amplification of viral or control RNA targets and LbCas12a for the trans-cleavage assay. A target amplification step is performed using the TwistAmp68 ® Basic recombinase polymerase amplification (RPA) kit (TwistDx, Cambridge, United Kingdom) and RT was carried out by the addition of input RNA, M71 MuLV reverse transcriptase (NEB), and murine RNase inhibitor (NEB). Reactions were run for 30 min at 42° C. The RT-RPA was carried out in one step, with the same pair of primers for both reactions. Reactions were performed independently for N-gene, E-gene, and RNase P.

LbCas12a trans-cleavage assays were performed similar to those described in Chen et al. (Science 360:436-439 (2018)). 50 nM LbCas12a (available from NEB) was pre-incubated with 62.5 nM gRNA in 1× NEBuffer 2.1 for 30 minutes at 37° C. After formation of the RNA-protein complex, the lateral flow cleavage reporter (/56-FAM/TTATTATT/3Bio/, IDT) was added to the reaction at a final concentration of 500 nM. RNA-protein complexes were used immediately or stored at 4° C. for up to 24 hours before use. After completion of the pre-amplification step, 2 μL of amplicon was combined with 18 μL of LbCas12a-gRNA complex and 80 μL of 1× NEBuffer 2.1. The 100 μL LbCas12a trans-cleavage assay was allowed to proceed for 10 minutes at 37° C.

A lateral flow strip (Milenia HybriDetect 1, TwistDx) was then added to the reaction tube and a result was visualized after approximately 2-3 minutes. A single band, close to the sample application pad indicated a negative result, whereas a single band close to the top of the strip or two bands indicated a positive result.

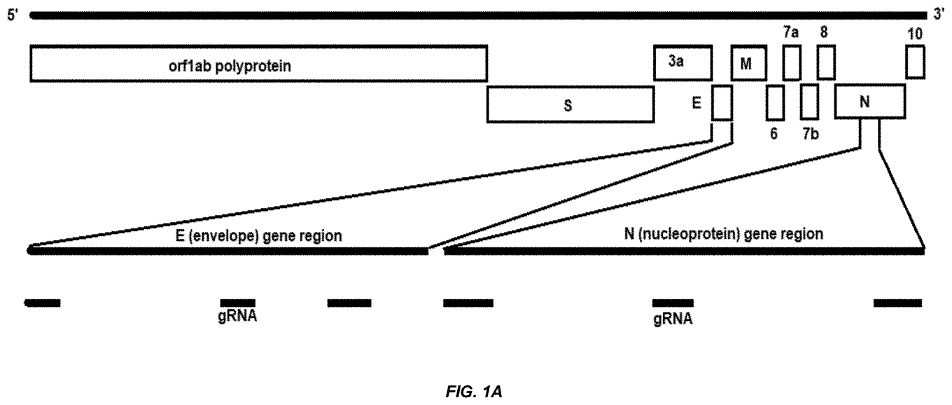

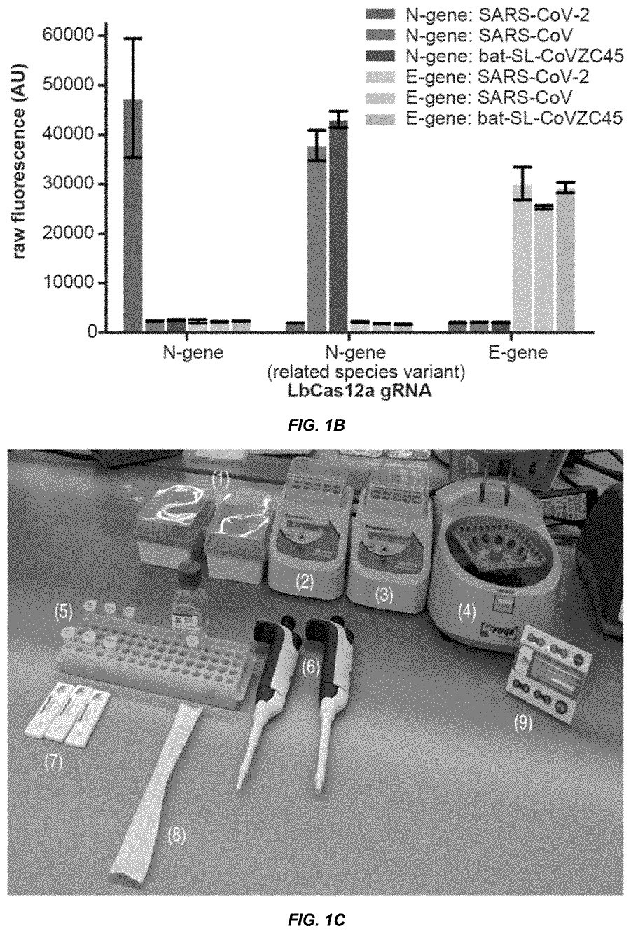

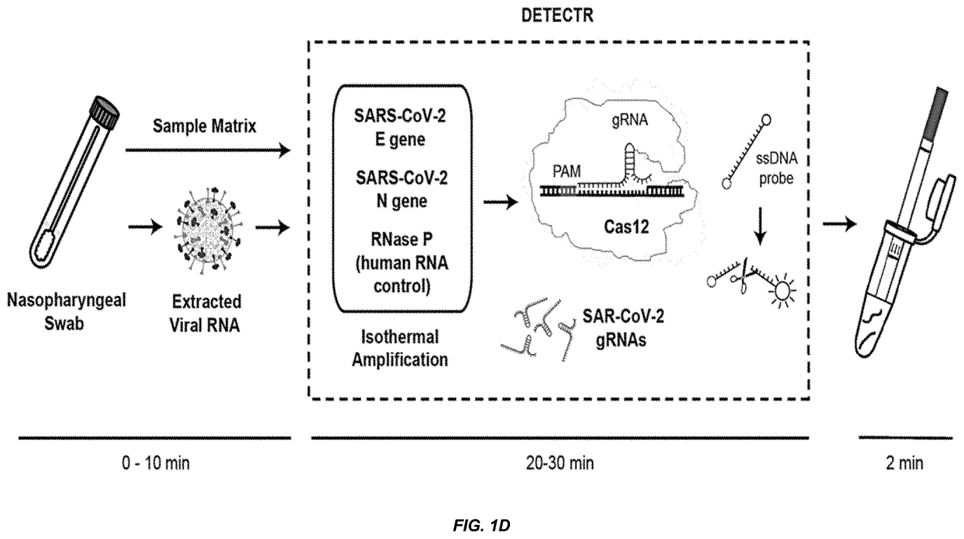

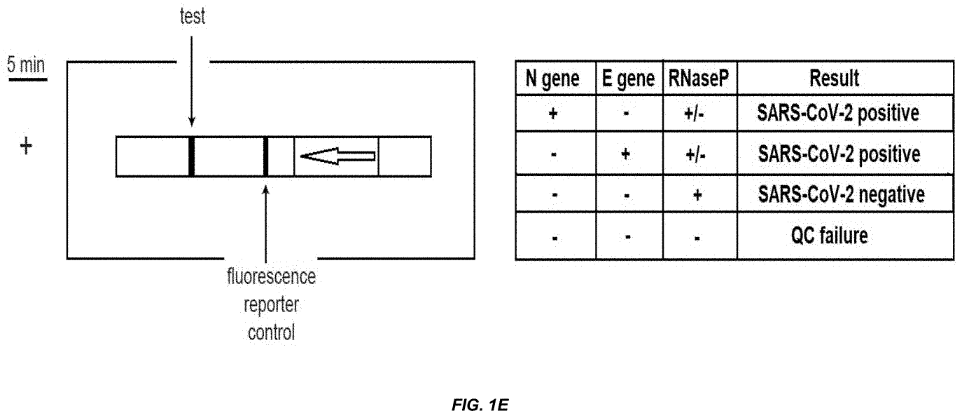

Primer Design. Primers were first designed that targeted the E (envelope) and N (nucleoprotein) genes of SARS-CoV-2 (e.g., see FIG. 1A). The primers amplify regions that overlap the World Health Organization (WHO) assay (E gene region) and US CDC assay (N2 region in the N gene), but are modified to meet design requirements for RPA (e.g., longer 30-34 nt primers). Next, Cas12 gRNAs were designed to detect three SARS-like coronaviruses (SARS-CoV-2 (accession NC 045512), bat SARS-like coronavirus (bat-SL-CoVZC45, accession MG772933) and SARS-CoV (accession NC_004718)) in the E gene and specifically detect only SARS-CoV-2 in the N gene (See FIG. 1A). This design is similar to those used by the WHO and US CDC assays, which use multiple amplicons with probes that are either specific to SARS-CoV-2 or are capable of identifying related SARS-like coronaviruses.

SARS-CoV-2-Dectection assay. Using synthetic, in vitro-transcribed (IVT) SARS-CoV-2 RNA gene targets in nuclease-free water, it was demonstrated that CRISPR-Cas12-based detection can distinguish SARS-CoV-2 with no cross-reactivity for related coronavirus strains using N gene gRNA and with expected cross-reactivity for E gene gRNA (see FIG. 1B). Conditions were then optimized for the SARS-CoV-2-Dectection assay on the E gene, N gene and human RNase P gene as a control, which consists of an RPA reaction at 40-42° C. for 20-30 min and Cas12 detection reaction at 37° C. for 10 min. The SARS-CoV-2-Dectection assay can be run in approximately 30-40 min and visualized on a lateral flow strip (see FIG. 1C-D). The SARS-CoV-2-Detection assay is considered positive if there is detection of both the E and N genes or presumptive positive if there is detection of either the E or N gene (see FIG. 1E). This interpretation is consistent with that of current US FDA EUA guidance and recently approved point-of-care diagnostics under the EUA.

TABLE 3
Comparison of the DETECTR LAMP/Cas12 and RPA/Cas12 assays
with the CDC qRT-PCR assay for detection of SARS-CoV-2

| | SARS-CoV-2 | CDC SARS-CoV2 |
|---|---|---|
| | DETECTR RPA/Cas12 | qRT-PCR |
| Target | N gene & E gene* | N-gene (3 amplicons, |
| | | N1, N2, and N3) |
| Sample control | RNase P | RNase P |
| Assay reaction time | 30 min | 120 min |
| (approximate) | | |
| Assay sample-to- | 30 min (sample matrix) | 4 h (RNA extract) |
| result time | 1 h (RNA extract) | |
| (approximate) | | |
| Assay components | RT-RPA (42° C., 20 min) | UDG digestion (25° C., |
| | Cas12 (37° C., 10 min) | 2 min), reverse |
| | Lateral flow (RT, 2 min) | transcription (50° C., 15 |
| | | min), denature (95° C., |
| | | 2 min) amplification, (95° |
| | | C., 3 sec; 55° C. 30 sec; |
| | | 45 cycles) |
| Bulky | No | Yes |
| instrumentation | | |
| required | | |
| FDA EUA approval | No | Yes |

*E gene primers target same amplicon region as in the WHO protocol; N gene primers target same N2 amplicon region as in the CDC protocol

Visualization of the Cas12 detection reaction. Visualization of the Cas12 detection reaction is achieved using a FAM-biotin reporter molecule and lateral flow strips designed to capture labeled nucleic acids (see FIG. 2). Uncleaved reporter molecules are captured at the first detection line (control line), whereas indiscriminate Cas12 cleavage activity generates a signal at the second detection line (test line). The SARS-CoV-2 DETECTR assay is considered positive if there is detection of both the E and N genes or presumptive positive if there is detection of either the E or N gene (see FIG. 1D). This interpretation is consistent with that of current US FDA EUA guidance and recently approved point-of-care diagnostics under the EUA16. Visualization of the Cas12 detection reaction is achieved using a FAM-biotin reporter molecule and lateral flow strips designed to capture labeled nucleic acids (see FIG. 2).

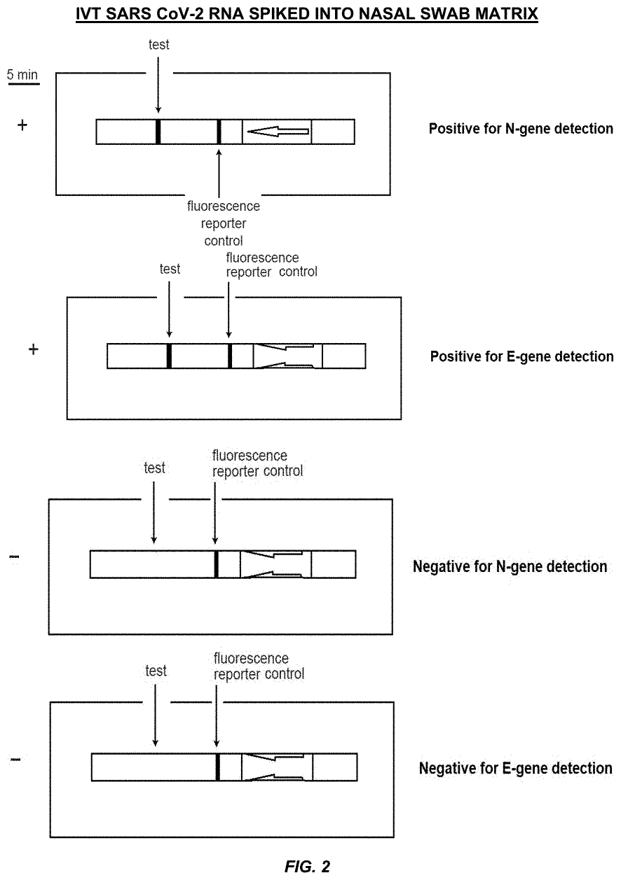

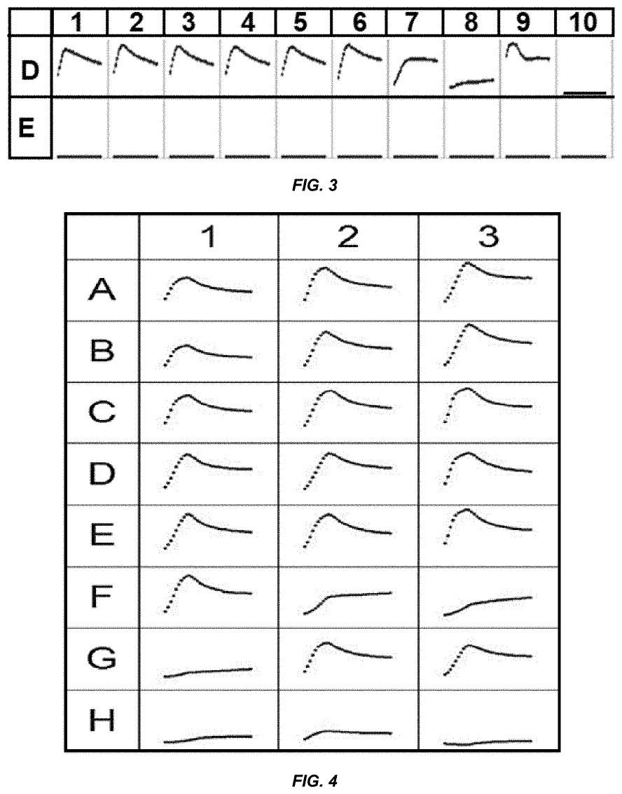

RT-RPA-SARS-2. The fluorescence-based LbCas12a trans-cleavage assays are performed as described above. A total of 2 μl of RPA amplicon is combined with 18 μl of LbCas12a-gRNA complex in an assay plate and monitored for fluorescence using a Tecan plate reader. The results are presented in Table 4; and FIGS. 3-4.

TABLE 4
RT-RPA-SARS-CoV-2 Data

| | | Fluorescence | | | |
|---|---|---|---|---|---|
| IVT-N2-RNA | | reading time | Incubation | | |
| concentration | Plate well | point | temperature | Fluorescence | Positive |
| (copies/ul) | position | (Min/sec) | when reading | unit | or Negative |
| 1000 | D1 | 13:50 | 37 | 8192 | Positive |
| 500 | D2 | 13:50 | 37 | 8627 | Positive |
| 250 | D3 | 13:50 | 37 | 8525 | Positive |
| 100 | D4 | 13:50 | 37 | 8273 | Positive |
| 50 | D5 | 13:50 | 37 | 8154 | Positive |
| 25 | D6 | 13:50 | 37 | 8108 | Positive |
| 10 | D7 | 13:50 | 37 | 4906 | Positive |
| 7 | D8 | 13:50 | 37 | 2152 | Negative |
| Positive control | D9 | 13:50 | 37 | 9178 | Positive |
| Negative control | D10 | 13:50 | 37 | 305 | Negative |
| Empty well | D11 | 13:50 | 37 | 302 | Negative |
| E gene | | Fluorescence | | | |
| synthetic DNA | | reading time | Incubation | | |
| concentration | Plate well | point | temperature | Fluorescence | Positive |
| (copies/ul) | position | (Min:sec) | when reading | unit | or negative |
| 10\hat{ }5 | A1 | 13:50 | 37 | 13:50 | Positive |
| 10\hat{ }5 | A2 | 13:50 | 37 | 13:50 | Positive |
| 10\hat{ }5 | A3 | 13:50 | 37 | 13:50 | Positive |
| 10\hat{ }4 | B1 | 13:50 | 37 | 13:50 | Positive |
| 10\hat{ }4 | B2 | 13:50 | 37 | 13:50 | Positive |
| 10\hat{ }4 | B3 | 13:50 | 37 | 13:50 | Positive |
| 10\hat{ }3 | C1 | 13:50 | 37 | 13:50 | Positive |
| 10\hat{ }3 | C2 | 13:50 | 37 | 13:50 | Positive |
| 10\hat{ }3 | C3 | 13:50 | 37 | 13:50 | Positive |
| 10\hat{ }2 | D1 | 13:50 | 37 | 13:50 | Positive |
| 10\hat{ }2 | D2 | 13:50 | 37 | 13:50 | Positive |
| 10\hat{ }2 | D3 | 13:50 | 37 | 13:50 | Positive |
| 10 | E1 | 13:50 | 37 | 13:50 | Positive |
| 10 | E2 | 13:50 | 37 | 13:50 | Positive |
| 10 | E3 | 13:50 | 37 | 13:50 | Positive |
| 5 | F1 | 13:50 | 37 | 13:50 | Positive |
| 5 | F2 | 13:50 | 37 | 13:50 | Negative |
| 5 | F3 | 13:50 | 37 | 13:50 | Negative |
| 1 | G1 | 13:50 | 37 | 13:50 | Negative |
| 1 | G2 | 13:50 | 37 | 13:50 | Positive |
| 1 | G3 | 13:50 | 37 | 13:50 | Positive |
| Negative-control | H1 | 13:50 | 37 | 13:50 | Negative |
| Negative-control | H2 | 13:50 | 37 | 13:50 | Negative |
| Negative-control | H3 | 13:50 | 37 | 13:50 | Negative |

RT-RPA-SARS-CoV-2 RT Performance. SARS-CoV-2 assays were performed using RPA for pre-amplification of viral or control RNA targets and a Cas12 protein for the trans-cleavage assay. A target amplification step was performed using the TwistAmp68 @ Basic recombinase polymerase amplification (RPA) kit (TwistDx, Cambridge, United Kingdom) and RT was carried out by the addition of input RNA, one of three reverse transcriptases, and murine RNase inhibitor (NEB). The three reverse transcriptases were M71 MuLV reverse transcriptase (NEB), SSII reverse transcriptase, and RevertAid reverse transcriptase (Thermo Scientific). Reactions were run for 1 hour at 37° C. The RT-RPA was carried out in one step, with the same pair of primers for both reactions. Reactions were performed independently for N-gene, E-gene, and RNase P.

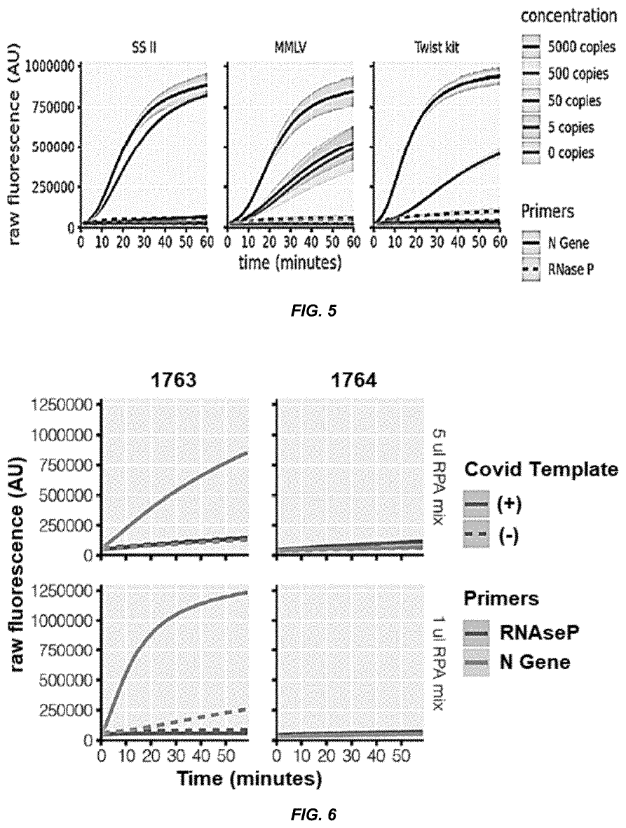

Cas12 trans-cleavage assays were performed similar to those described in Chen et al. (Science 360:436-439 (2018)). 40 nM Cas12 protein was pre-incubated with 40 nM N-gene gRNA in buffer at 37° C. After formation of the RNA-protein complex, the reporter was added to the reaction at a final concentration of 100 nM. RNA-protein complexes were used immediately or stored at 4° C. for up to 24 hours before use. After completion of the pre-amplification step, 1 μL of amplicon at various concentrations (5000 cp/μl, 500 cp/μl, 50 cp/μl, 5 cp/μl, 0 cp/μl) was combined with 5 μL of Cas12-gRNA complex and 14 μL of buffer. The 20 μL Cas12 trans-cleavage assay was allowed to proceed for 1 hour at 37° C. and fluorescence was monitored on a QuantStudio 5. The results are presented in FIG. 5. All three reverse transcriptases produced consistent results, with M71 MuLV reverse transcriptase having the lowest limit of detection under the assay conditions tested.

RT-RPA-SARS-CoV-2 Reagent Specificity. SARS-CoV-2 assays were performed using RPA for pre-amplification of viral or control RNA targets and a Cas12 protein for the trans-cleavage assay. A target amplification step was performed using the TwistAmp68 ® Basic recombinase polymerase amplification (RPA) kit (TwistDx, Cambridge, United Kingdom) and RT was carried out by the addition of input RNA, M71 MuLV reverse transcriptase (NEB), and murine RNase inhibitor (NEB). Reactions were run for 1 hour at 37° C. The RT-RPA was carried out in one step, with the same pair of primers for both reactions. Reactions were performed independently for N-gene, E-gene, and RNase P.

Cas12 trans-cleavage assays were performed similar to those described in Chen et al. (Science 360:436-439 (2018)). 40 nM Cas12 protein was pre-incubated with 40 nM N-gene gRNA 1763 or E-gene gRNA 1764 in buffer at 37° C. After formation of the RNA-protein complex, the reporter was added to the reaction at a final concentration of 100 nM. RNA-protein complexes were used immediately or stored at 4° C. for up to 24 hours before use. After completion of the pre-amplification step, 1 μL or 5 μL of amplicon at about 10,000 cp/μl was combined with 5 μL of Cas12-gRNA complex and 14 μL or 10 μL of buffer, respectively. The 20 μL Cas12 trans-cleavage assay was allowed to proceed for 1 hour at 37° C. and fluorescence was monitored on a QuantStudio 5. The results are presented in FIG. 6. Strong amplification was detected when the correct (N-gene) gRNA, primer sets, and templates were present. Little to no cross-reactivity occurred with RNAse P primers, E-gene gRNA, or no template controls.

RT-RPA-SARS-CoV-2 RPA:DETECTR Ratio Optimization. SARS-CoV-2 assays were performed using RPA for pre-amplification of viral or control RNA targets and a Cas12 protein for the trans-cleavage assay. A target amplification step was performed using the TwistAmp68 ® Basic recombinase polymerase amplification (RPA) kit (TwistDx, Cambridge, United Kingdom) and RT was carried out by the addition of input RNA, M71 MuLV reverse transcriptase (NEB), and murine RNase inhibitor (NEB). The amount of dNTP in the reaction was varied from 1.6 mM to 0 mM (1.6 mM, 1.4 mM, 1.2 mM, 1 mM, 0.8 mM, 0.6 mM, 0.4 mM, 0 mM). Reactions were run for 1 hour at 37° C. The RT-RPA was carried out in one step, with the same pair of primers for both reactions. Reactions were performed independently for N-gene, E-gene, and RNase P.

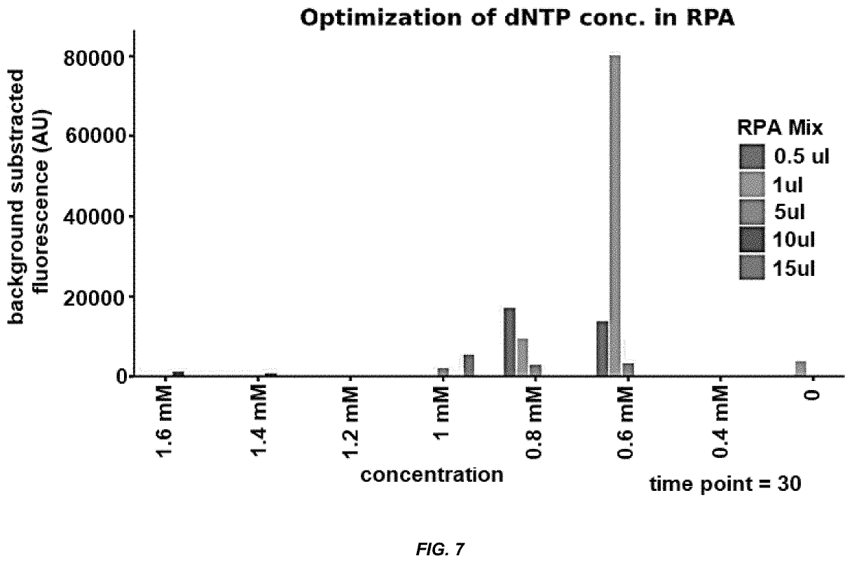

Cas12 trans-cleavage assays were performed similar to those described in Chen et al. (Science 360:436-439 (2018)). 40 nM Cas12 protein was pre-incubated with 40 nM N-gene gRNA in buffer at 37° C. After formation of the RNA-protein complex, the reporter was added to the reaction at a final concentration of 100 nM. RNA-protein complexes were used immediately or stored at 4° C. for up to 24 hours before use. After completion of the pre-amplification step, various amounts (0.5 μl, 1 μl, 5 μl, 10 μl, 15 μl) of amplicon at about 10,000 cp/μl were combined with 5 μL of Cas12-gRNA complex and a volume of buffer to bring the total assay volume up to 20 μL. The 20 μL Cas12 trans-cleavage assay was allowed to proceed for 1 hour at 37° C. and fluorescence was monitored on a QuantStudio 5. The results are presented in FIG. 7. Under the conditions tested, DETECTR signal was strongest when 1 μl of RPA amplicon mix having 0.6 mM dNTP was added to the Cas12 trans-cleavage assay.

A number of embodiments of the disclosure have been described. Nevertheless, it will be understood that various modifications may be made without departing from the spirit and scope of the disclosure. Accordingly, other embodiments are within the scope of the following claims.

1. A recombinase polymerase amplification (RPA) method to detect SARS-CoV-2 in a sample, comprising:
performing a RT-RPA reaction to generate isothermal amplification products by incubating at an elevated temperature a first reaction mixture comprising reverse transcriptase, extracted RNA from a sample, primers that are specific to a gene of SARS-CoV-2, and recombinase and polymerase enzymes, wherein the gene is the N-gene, or E-gene of SARS-CoV-2;
performing a trans-cleavage assay by incubating at an elevated temperature a second reaction mixture comprising the amplification products from the previous step, a programmable nuclease that has been complexed with gRNA specific to corresponding gene sequences of SARS-CoV-2, and a single-stranded detector nucleic acid comprising a detection moiety, wherein the detector nucleic acid is trans-cleaved by the programmable nuclease if SARS-CoV-2 gene amplification products are present in the second reaction mixture; and
detecting whether SARS-CoV-2 is the sample based upon detecting trans-cleaved detector nucleic acid fragments.

2. The RPA method of claim 1, wherein the recombinase and polymerase enzymes include one or more of the following T4 UvsX protein, T4 UvsY protein, T4 gp32, Bacillus subtilis DNA polymerase I and Staphylococcus aureus polymerase.

3. The RPA method of claim 1, wherein the first reaction mixture is incubated at 40-42° C. for 20-30 min.

4. The RPA method of claim 1, wherein the primers target the N2 region in the N gene of SARS-CoV-2.

5. The RPA method of claim 1, wherein the primers have the sequences of (1) or (2):
(SEQ ID NO: 5)
(1) GAAGAGACAGGTACGTTAATAGTTAATAGCGT
and/or
(SEQ ID NO: 23)
ACGTTAACAATATTGCAGCAGTACGCACACA;
or
(SEQ ID NO: 7)
(2) ACAAGGAACTGATTACAAACATTGGCCGCAAA
and/or
(SEQ ID NO: 8)
TTCCATGCCAATGCGCGACATTCCGAAGAA.

6. The RPA method of claim 1, wherein the RNA is extracted from an environmental sample or a sample from a subject.

7. The RPA method of claim 6, wherein the sample is a nasopharyngeal and/or oropharyngeal swab sample obtained from a human patient.

8. (canceled)

9. The RPA method of claim 1, wherein the RT-RPA reaction and the trans-cleavage assay are performed as a single-tube reaction.

10. The RPA method of claim 1, wherein the programmable nuclease is a Cas12 nuclease, a Cas13 nuclease, or a Cas14 nuclease.

11. The RPA method of claim 10, wherein the programmable nuclease is a Cas12a nuclease.

12. The RPA method of claim 10, wherein the polypeptide sequence of the programmable nuclease has at least 85% sequence identity to SEQ ID NO:1, 2, 3, or 4.

13. The RPA method of claim 12, wherein the polypeptide sequence of the programmable nuclease has at least 95% sequence identity to SEQ ID NO:4.

14. The RPA method of claim 1, wherein the gRNA is specific to the N-gene, E-gene, or human Rnase P gene of SARS-CoV-2.

15. The RPA method of claim 14, wherein the gRNA is specific to the N-gene of SARS-CoV-2 and comprises the sequence of:
(SEQ ID NO: 9)
(a) UAAUUUCUACUAAGUGUAGAUCCCCCAGCGCUUCAGCGUUC.

16. The RPA method of claim 14, wherein the gRNA is specific to the E-gene of SARS-CoV-2 and comprises the sequence of (c) or (d):
(SEQ ID NO: 10)
(c) UAAUUUCUACUAAGUGUAGAUUUGCUUUCGUGGUAUUCUUG;
or
(SEQ ID NO: 11)
(d) UAAUUUCUACUAAGUGUAGAUGUGGUAUUCUUGCUAGUUAC.

17. The RPA method of claim 1, wherein the detector nucleic acid comprises a fluorescence detection moiety.

18. The RPA method of claim 17, wherein the fluorescence detection molecule is fluorescein or a chemical derivative thereof.

19. The RPA method of claim 1, wherein the detector nucleic acid comprises a biotin moiety.

20. The RPA method of claim 1, wherein the detector nucleic acid comprises the sequence of (e) or (f):
(e) 5′-(FAM)-TTATTATT-(BHQ-1)-3′, wherein FAM is fluorescein and wherein BHQ-1 is a black hole quencher dye; or
(f) 5′-(FAM)-TTATTATT-(Bio)-3′, wherein FAM is fluorescein and wherein Bio is biotin.

21. The RPA method of claim 1, wherein the trans-cleaved detector nucleic acid fragments are detected using a lateral flow assay.

22. The RPA method of claim 1, wherein the trans-cleaved detector nucleic acid fragments are detected using a microplate reader.

23. A nucleic acid detection method comprising:
a nucleic acid amplification reagent and primers for recombinase polymerase amplification (RPA), wherein the primers comprise sequences that are at least 85%, at least 90%, at least 92%, at least 95%, or at least 98% identical to
(SEQ ID NO: 5)
(1) GAAGAGACAGGTACGTTAATAGTTAATAGCGT
(SEQ ID NO: 23)
(2) ACGTTAACAATATTGCAGCAGTACGCACACA;
(SEQ ID NO: 7)
(3) ACAAGGAACTGATTACAAACATTGGCCGCAAA
or
(SEQ ID NO: 8)
(4) TTCCATGCCAATGCGCGACATTCCGAAGAA,
wherein the primers amplify target nucleic acid of SARS-CoV-2,
contacting a sample suspected of containing SARS-CoV-2 nucleic acids with the nucleic acid amplification reagent and primers under conditions to amplify target nucleic acids;
providing a CRISPR-associated (Cas) enzyme with trans cleavage activity and a guide CRISPR RNA (gRNA) comprising a guide sequence, wherein the guide sequence is configured to bind to amplified target nucleic acids; and
providing a plurality of probes, each probe comprising an oligonucleotide element labeled with a detectable label, wherein the probe is configured to be cleaved by the Cas enzyme when the guide sequence binds to amplified target nucleic acids to generate a detectable signal or a detectable molecule;
contacting the sample suspected of containing SARS-CoV-2 nucleic acids under conditions with the CRISPR-associated enzyme, the gRNA and the plurality of probes to facilitate CRISPR-Cas trans cleavage.

24. The detection system of claim 23, wherein a first end of the oligonucleotide element in the probe is linked to a fluorophore; a second end of the oligonucleotide element in the probe is linked to a quencher such that the fluorophore produces a detectable signal upon cleavage of the oligonucleotide element to release the quencher.

25. The detection method of claim 23, wherein the probe comprises FAM-TTATT-3IABkFQ or FAM-TTATTA(internal biotin)T-3IABkFQ.

26. The method of claim 23, wherein the sample is selected from the group consisting of saliva, respiratory secretions, exudate, blood, plasma, urine, stool, and sera.

27. A kit compartmentalized to contain reagents for carrying out the method of claim 1.


SEQUENCE LISTING (30 sequences)

SEQ ID NO: 1 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 2 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 3 - protein, 1129 aa, Alicyclobacillus acidoterrestris
MAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVDLGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLEELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHADLNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDI

SEQ ID NO: 4 - protein, 1228 aa, Lachnospiraceae bacterium
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 5 - DNA, 32 bp, Artificial Sequence
  Primer sequence
gaagagacaggtacgttaatagttaatagcgt

SEQ ID NO: 6 - DNA, 30 bp, Artificial Sequence
  Primer sequence
cgttaacaatattgcagcagtacgcacaca

SEQ ID NO: 7 - DNA, 32 bp, Artificial Sequence
  Primer sequence
acaaggaactgattacaaacattggccgcaaa

SEQ ID NO: 8 - DNA, 30 bp, Artificial Sequence
  Primer sequence
ttccatgccaatgcgcgacattccgaagaa

SEQ ID NO: 9 - RNA, 41 nt, Artificial Sequence
  gRNA sequence
uaauuucuacuaaguguagaucccccagcgcuucagcguuc

SEQ ID NO: 10 - RNA, 41 nt, Artificial Sequence
  gRNA sequence
uaauuucuacuaaguguagauuugcuuucgugguauucuug

SEQ ID NO: 11 - RNA, 41 nt, Artificial Sequence
  gRNA sequence
uaauuucuacuaaguguagaugugguauucuugcuaguuac

SEQ ID NO: 12 - DNA, 20 bp, Artificial Sequence
  Primer sequence
ttacaaacattggccgcaaa

SEQ ID NO: 13 - DNA, 18 bp, Artificial Sequence
  Primer sequence
gcgcgacattccgaagaa

SEQ ID NO: 14 - DNA, 23 bp, Artificial Sequence
  Primer sequence
acaatttgcccccagcgcttcag

SEQ ID NO: 15 - DNA, 20 bp, Artificial Sequence
  Primer sequence
cgaggtgggacttcagcatg

SEQ ID NO: 16 - DNA, 20 bp, Artificial Sequence
  Primer sequence
gagcggctgtctccacaagt

SEQ ID NO: 17 - DNA, 23 bp, Artificial Sequence
  Primer sequence
ttctgacctgaaggctctgcgcg

SEQ ID NO: 18 - DNA, 26 bp, Artificial Sequence
  Primer sequence
acaggtacgttaatagttaatagcgt

SEQ ID NO: 19 - DNA, 22 bp, Artificial Sequence
  Primer sequence
atattgcagcagtacgcacaca

SEQ ID NO: 20 - DNA, 26 bp, Artificial Sequence
  Primer sequence
acactagccatccttactgcgcttcg

SEQ ID NO: 21 - DNA, 31 bp, Artificial Sequene
  Primer sequence
acgttaacaatattgcagcagtacgcacaca

SEQ ID NO: 22 - DNA, 30 bp, Artificial Sequence; identical to SEQ ID NO: 8
  Primer sequence

SEQ ID NO: 23 - DNA, 30 bp, Artificial Sequence
  Primer sequence
gaattcggcacgaggtgggacttcagcatg

SEQ ID NO: 24 - DNA, 32 bp, Artificial Sequence
  Primer sequence
aatagccaaggtgagcggctgtctccacaagt

SEQ ID NO: 25 - DNA, 30 bp, Artificial Sequence
  Primer sequence
ggaccaggaactaatcagacaaggaactga

SEQ ID NO: 26 - DNA, 30 bp, Artificial Sequence
  Primer sequence
aggtcaaccacgttcccgaaggtgtgactt

SEQ ID NO: 27 - RNA, 30 nt, Artificial Sequence
  Primer sequence
ggcagacgugguccagaacaaacccaagga

SEQ ID NO: 28 - DNA, 31 bp, Artificial Sequence
  Primer sequence
taggtcaaccacgttcccgaaggtgtgactt

SEQ ID NO: 29 - DNA, 181 bp, Artificial Sequence
  Oligonucleotide template RNase-P-oligo
taatacgactcactataggaattcggcacgaggtgggacttcagcatggcggtgtttgcagatttggacctgcgagcgggttctgacctgaaggctctgcgcggacttgtggagacagccgctcaccttggctattcagttgttgctatcaatcatatcgttgactttaaggaaaagaaac

SEQ ID NO: 30 - DNA, 186 bp, Artificial Sequence
  Oligonucleotide template N2-oligo
taatacgactcactatagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtg